(19) United States
(12) Patent Application Publication
(10) Pub. No.: US 2025/0331854 A1
(43) Pub. Date: Oct. 30, 2025

(54) SURGICAL STAPLER WITH FIRING LOCKOUT FEATURE COUPLED TO END EFFECTOR JAW

(71) Applicant: Cilag GmbH International, Zug (CH)

(72) Inventors: Joseph T. Mozloom, JR., Cincinnati, OH (US); Paul Moubarak, West Chester, OH (US); Andrew R. Conway, Cincinnati, OH (US); Zhifan F. Huang, Mason, OH (US); Michael D. Auld, Milford, OH (US); Mark T. Larson, Cincinnati, OH (US); Jason T. Clement, Roseville, MN (US); Seth D. Holdmeyer, Sharonville, OH (US)

(21) Appl. No.: 18/650,653

(22) Filed: Apr. 30, 2024

(51) Int. Cl. A61B 17/072; A61B 17/29

(52) U.S. Cl. CPC A61B 17/07207; A61B 2017/07271; A61B 2017/07278; A61B 2017/07285; A61B 2017/2946

(57) ABSTRACT

An apparatus (1000) includes a shaft (600A) and an end effector (1200, 2200) configured to receive a staple cartridge (210) having staples and a sled (1210a, 2210a). The end effector includes a knife (1206, 2206), an anvil jaw (204, 1204), a cartridge jaw (1208, 2208), and a blocker (1255, 2255) coupled to the cartridge jaw. The blocker is vertically translatable relative to the cartridge jaw and the knife between a raised and lowered positions. The blocker in the raised position is configured to directly contact and thereby inhibit distal advancement of the knife, and the blocker in the lowered position is configured to permit distal advancement of the. The blocker is configured to assume the raised position in the absence of an unspent staple cartridge and the lowered position in the presence of an unspent staple cartridge.

### BACKGROUND

In some settings, endoscopic surgical instruments may be preferred over traditional open surgical devices to minimize the size of the surgical incision as well as post-operative recovery time and complications. Consequently, some endoscopic surgical instruments may be suitable for placement of a distal end effector at a desired surgical site through the cannula of a trocar. These distal end effectors may engage tissue in a number of ways to achieve a diagnostic or therapeutic effect (e.g., endocutter, grasper, cutter, stapler, clip applier, access device, drug/gene therapy delivery device, and energy delivery device using ultrasound, RF, laser, etc.). Endoscopic surgical instruments may include a shaft that extends proximally from the end effector to a handle portion that is manipulated by the clinician, or alternatively to a robot. Such a shaft may enable insertion to a desired depth and rotation about the longitudinal axis of the shaft, thereby facilitating positioning of the end effector within the patient. Positioning of an end effector may be further facilitated through inclusion of one or more articulation joints or features, enabling the end effector to be selectively articulated or otherwise deflected relative to the longitudinal axis of the shaft.

Examples of endoscopic surgical instruments include surgical staplers. Some such staplers are operable to clamp down on layers of tissue, cut through the clamped layers of tissue, and drive staples through the layers of tissue to substantially seal the severed layers of tissue together near the severed ends of the tissue layers. Such endoscopic surgical staplers may also be used in open procedures and/or other non-endoscopic procedures. By way of example only, a surgical stapler may be inserted through a thoracotomy and thereby between a patient's ribs to reach one or more organs in a thoracic surgical procedure that does not use a trocar as a conduit for the stapler. Such procedures may include the use of the stapler to sever and close a vessel leading to an organ, such as a lung. For instance, the vessels leading to an organ may be severed and closed by a stapler before removal of the organ from the thoracic cavity. Of course, surgical staplers may be used in various other settings and procedures.

The surgical stapling features of the present disclosure seek to inhibit firing of a surgical stapler end effector when the end effector is loaded with a spent staple cartridge that has already been fired, and/or when a staple cartridge is entirely absent from the end effector. Specifically, such features of the present disclosure place the end effector in a lockout state that inhibits firing in either of such scenario. While various kinds of surgical staplers and associated components have been made and used, it is believed that no one prior to the inventor(s) has made or used the invention described in the appended claims.

### BRIEF DESCRIPTION OF THE DRAWINGS

The accompanying drawings, which are incorporated in and constitute a part of this specification, illustrate examples of the invention, and, together with the general description of the invention given above, and the detailed description of the examples given below, serve to explain the principles of the present invention.

FIG. 1 is a perspective view of an illustrative surgical instrument having a housing, a shaft assembly, an articulation joint, and an end effector;

FIG. 2 is a partial perspective view of the surgical instrument of FIG. 1, with select components omitted from view to reveal portions of a cable articulation subsystem, a knife firing subsystem, and a roll subsystem of the surgical instrument;

FIG. 3 is an enlarged perspective view of the end effector and the articulation joint of the surgical instrument of FIG. 1;

FIG. 4 is an exploded view of a distal end portion of the surgical instrument of FIG. 1;

FIG. 5 is an enlarged perspective view of a knife of the end effector of the surgical instrument of FIG. 1;

FIG. 6 is an end view of the end effector of FIG. 3;

FIG. 7 is an enlarged perspective view of the end effector and the articulation joint of FIG. 3, with an anvil of the end effector omitted;

FIG. 8A is a side cross-sectional view of a distal end portion of the surgical instrument of FIG. 1, depicting the anvil in an open position;

FIG. 8B is a side cross-sectional view of the distal end portion of the surgical instrument of FIG. 1, depicting the anvil in a grasping position with the knife partially advanced;

FIG. 8C is a side cross-sectional view of the distal end portion of the surgical instrument of FIG. 1, depicting the anvil in a clamping position with the knife partially advanced;

FIG. 8D is a side cross-sectional view of the distal end portion of the surgical instrument of FIG. 1, depicting the anvil in the clamping position with the knife fully advanced;

FIG. 9A is an enlarged side cross-sectional view of a proximal end portion of the end effector of the surgical instrument of FIG. 1, depicting the anvil in the open position;

FIG. 9B is an enlarged side cross-sectional view of the proximal end portion of the end effector of the surgical instrument of FIG. 1, depicting the anvil in a grasping position with the knife partially advanced;

FIG. 9C is an enlarged side cross-sectional view of the proximal end portion of the end effector of the surgical instrument of FIG. 1, depicting the anvil in a clamping position with the knife partially advanced;

FIG. 9D is an enlarged side cross-sectional view of the proximal end portion of the end effector of the surgical instrument of FIG. 1, depicting the anvil in the clamping position with the knife fully advanced;

FIG. 10 is an exploded perspective view of the articulation joint of the surgical instrument of FIG. 1;

FIG. 11 is an end view of the articulation joint of FIG. 10;

FIG. 12 is a cross-sectional view of a portion of the articulation joint of FIG. 10, taken along line 12-12 in FIG. 11;

FIG. 13 is a cross-sectional view of a portion of the articulation joint of FIG. 10, taken along line 13-13 in FIG. 11;

FIG. 14 is a perspective view of the distal end of the surgical instrument of FIG. 1, depicting the end effector articulated vertically and laterally with the anvil open;

FIG. 15 is a side view of the distal end of the surgical instrument of FIG. 1, depicting the end effector articulated vertically with the anvil closed;

FIG. 16 is a top view of the distal end of the surgical instrument of FIG. 1, depicting the end effector articulated laterally with the anvil closed;

FIG. 17 is an exploded perspective view of a portion of the surgical instrument of FIG. 1, depicting portions of the cable articulation subsystem, the knife firing subsystem, and the roll subsystem;

FIG. 18 is a top view of a proximal end of the surgical instrument of FIG. 1, depicting portions of the cable articulation subsystem, the knife firing subsystem, and the roll subsystem;

FIG. 19 is a perspective view of a shaft assembly, a differential, and a firing rod of the surgical instrument of FIG. 1;

FIG. 20 is an exploded perspective view of another end effector including a knife and a lockout assembly, among other components omitted from view;

FIG. 21 is an exploded perspective view of the lockout assembly of the end effector of FIG. 20;

FIG. 22A is a side cross-sectional view of a proximal end portion of the end effector of FIG. 20, depicting the lockout assembly in a locked configuration;

FIG. 22B is a side cross-sectional view of the proximal end portion of the end effector of FIG. 20, depicting a sled of an unspent staple cartidge transitioning the lockout assembly into an unlocked configuration when the unspent staple cartridge is seated in the end effector;

FIG. 22C is a side cross-sectional view of the proximal end portion of the end effector of FIG. 21, depicting the sled and knife translating beyond the lockout assembly;

FIG. 23 is an exploded perspective view of another end effector including a knife, a sled, and a lockout assembly;

FIG. 24 is an exploded perspective view of the knife and lockout assembly of the end effector of FIG. 23;

FIG. 25A is a side cross-sectional view of a proximal end portion of the end effector of FIG. 23, depicting the lockout assembly in a locked configuration;

FIG. 25B is a side cross-sectional view of the proximal end portion of the end effector of FIG. 23, depicting a sled of an unspent staple cartidge transitioning the lockout assembly into an unlocked configuration when the unspent staple cartridge is seated in the end effector;

FIG. 25C is a side cross-sectional view of the proximal end portion of the end effector of FIG. 23, depicting the sled and knife translating beyond the lockout assembly;

FIG. 26 is an exploded perspective view of another end effector including a knife, a cartridge jaw, and a lockout assembly, among other components omitted from view;

FIG. 27 is a top view of the cartridge jaw and lockout assembly of the end effector of FIG. 26;

FIG. 28 is an exploded perspective view of the lockout assembly of the end effector of FIG. 26;

FIG. 29A is a perspective view of the knife and lockout assembly of the end effector of FIG. 26, depicting the lockout assembly in a locked configuration;

FIG. 29B is a perspective view of the knife and lockout assembly of the end effector of FIG. 26, depicting the lockout assembly in an unlocked configuration;

FIG. 30A is a side cross-sectional view of the knife and lockout assembly of the end effector of FIG. 26, depicting the lockout assembly in the locked configuration;

FIG. 30B is a side cross-sectional view of the knife and lockout assembly of the end effector of FIG. 26, depicting the lockout assembly in the unlocked configuration;

FIG. 31 is a side cross-sectional view of the knife and lockout assembly of the end effector of FIG. 26, depicting the lockout assembly in the unlocked configuration and the knife translated distally;

FIG. 32 is an exploded perspective view of another end effector including a knife, a cartridge jaw, a sled, and a lockout assembly, among other components omitted from view;

FIG. 33A is a top view of a proximal end portion of the end effector of FIG. 32, depicting the lockout assembly in a locked configuration;

FIG. 33B is a top view of the proximal end portion of the end effector of FIG. 32, depicting the sled transitioning the lockout assembly into an unlocked configuration when an unspent staple cartridge is seated in the cartridge jaw;

FIG. 33C is a top view of the proximal end portion of the end effector of FIG. 32, depicting the sled and knife translating beyond the lockout assembly;

FIG. 34 is an exploded perspective view of a proximal end portion of another end effector including a knife, a cartridge jaw, and a lockout assembly, among other components omitted from view;

FIG. 35 is a perspective view of the proximal end portion of the end effector of FIG. 34, depicting the lockout assembly in a locked configuration;

FIG. 36A is a top view of the proximal end portion of the end effector of FIG. 34, depicting the lockout assembly in a locked configuration;

FIG. 36B is a top view of the proximal end portion of the end effector of FIG. 34, depicting a sled transitioning the lockout assembly into an unlocked configuration when an unspent staple cartridge is seated in the cartridge jaw; and

FIG. 36C is a top view of the proximal end portion of the end effector of FIG. 34, depicting the sled and knife translating beyond the lockout assembly.

### DETAILED DESCRIPTION

The following detailed description should be read with reference to the drawings, in which like elements in different drawings are identically numbered. The drawings, which are not necessarily to scale, depict selected versions and are not intended to limit the scope of the invention. The detailed description illustrates by way of example, not by way of limitation, the principles of the invention. This description will clearly enable one skilled in the art to make and use the invention, and describes several versions, adaptations, variations, alternatives and uses of the invention, including what is presently believed to be the best mode of carrying out the invention.

Numerous specific details are set forth to provide a thorough understanding of the overall structure, function, manufacture, and use of the versions as described in the specification and illustrated in the accompanying drawings. Well-known operations, components, and elements have not been described in detail so as not to obscure the versions described in the specification. The reader will understand that the versions described and illustrated herein are non-limiting examples, and thus it can be appreciated that the specific structural and functional details disclosed herein may be representative and illustrative. Variations and changes thereto may be made without departing from the scope of the claims.

The terms “comprise” (and any form of comprise, such as “comprises” and “comprising”), “have” (and any form of have, such as “has” and “having”), “include” (and any form of include, such as “includes” and “including”) and “contain” (and any form of contain, such as “contains” and “containing”) are open-ended linking verbs. As a result, a surgical system, device, or apparatus that “comprises,” “has,” “includes” or “contains” one or more elements possesses those one or more elements, but is not limited to possessing only those one or more elements. Likewise, an element of a system, device, or apparatus that “comprises,” “has,” “includes” or “contains” one or more features possesses those one or more features, but is not limited to possessing only those one or more features.

The terms “proximal” and “distal” are used herein with reference to a robotic platform manipulating the housing portion of the surgical instrument. The term “proximal” refers to the portion closest to the robotic platform and the term “distal” refers to the portion located away from the robotic platform. It will be further appreciated that, for convenience and clarity, spatial terms such as “vertical”, “horizontal”, “up”, and “down” may be used herein with respect to the drawings. However, surgical instruments are used in many orientations and positions, and these terms are not intended to be limiting and/or absolute.

Furthermore, the terms “about,” “approximately,” “substantially,” and the like as used herein in connection with any numerical values, ranges of values, and/or geometric/positional quantifications are intended to encompass the exact value(s) or quantification(s) referenced as well as a suitable tolerance that enables the referenced feature or combination of features to function for the intended purpose described herein. For example, “substantially parallel” encompasses nominally parallel structures, and “substantially equal” values encompass nominally equal values.

Furthermore, the use of “couple”, “coupled”, or similar phrases should not be construed as being limited to a certain number of components or a particular order of components unless the context clearly dictates otherwise.

### I. Overview of Illustrative Surgical Instrument

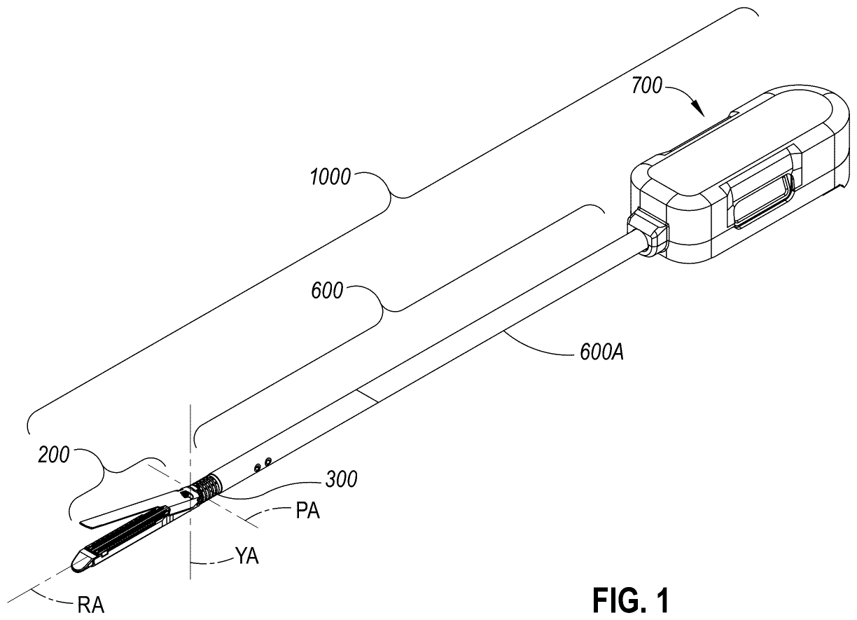

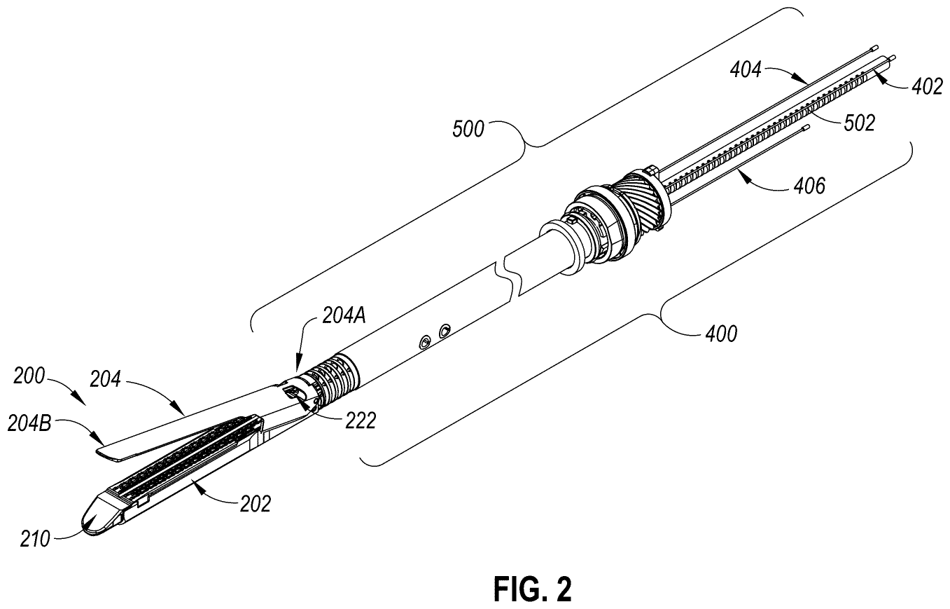

FIGS. 1-2 show an illustrative surgical instrument 1000 that is configured to grasp, clamp, incise, and seal patient tissue with staples. The surgical instrument 1000 comprises an end effector 200, an articulation joint 300 (also referred to as a “continuum joint”), an articulation drive subsystem 400 configured to articulate the end effector 200 via the articulation joint 300, a knife firing subsystem 500 configured to actuate the end effector 200 between various positions (e.g., an open position, a grasping position, and a clamping position) and to incise and staple patient tissue, a roll subsystem 600 configured to rotate the end effector 200 about a roll axis RA, and a housing 700.

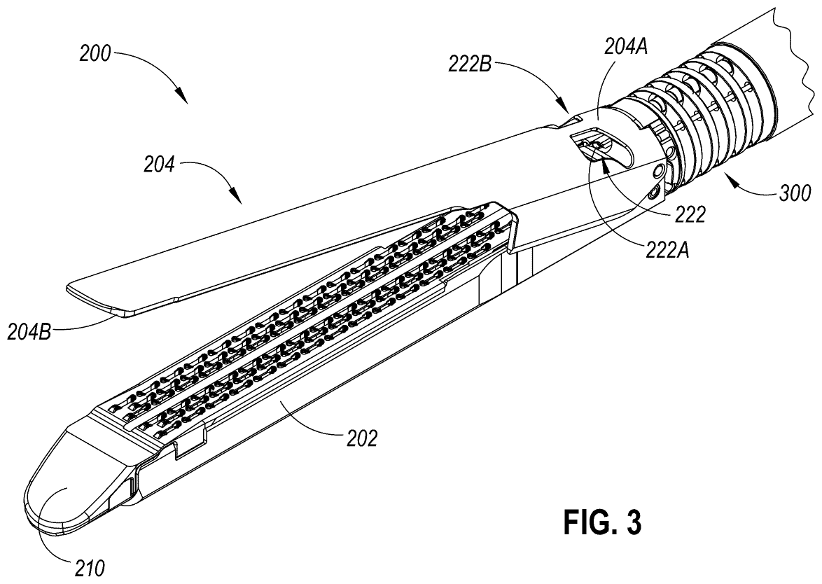

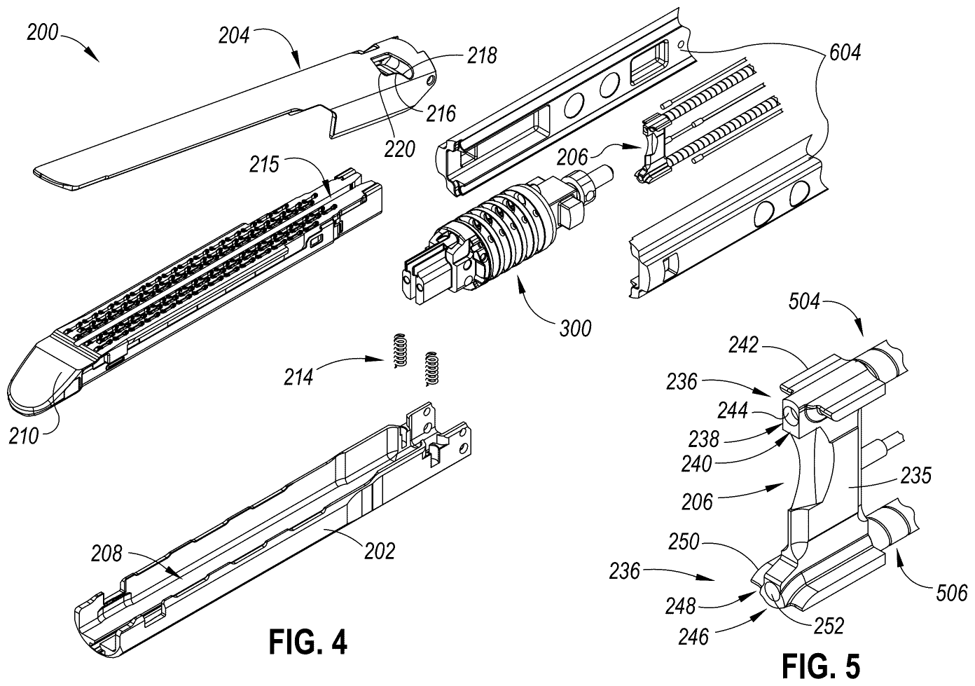

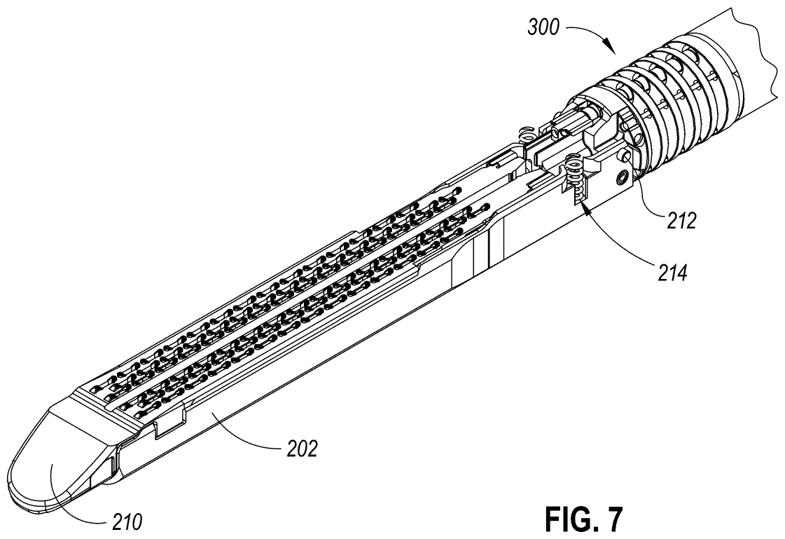

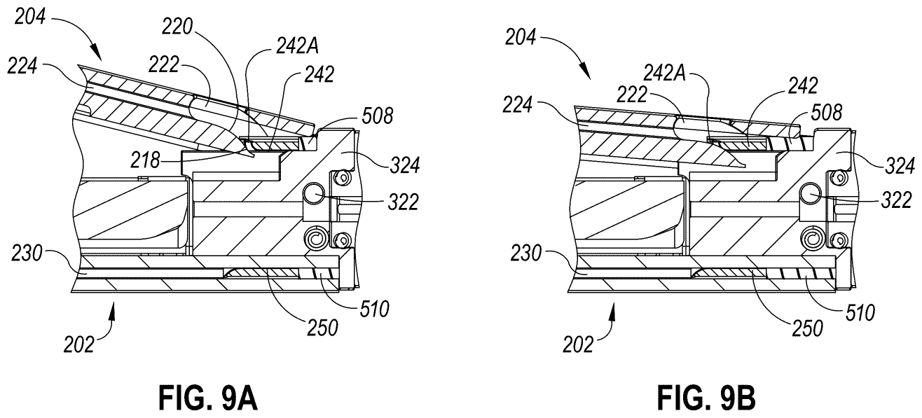

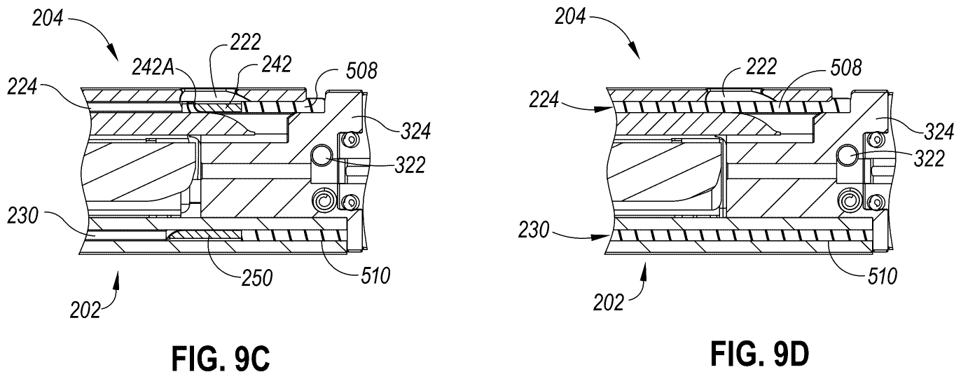

As shown best in FIGS. 3-4, the end effector 200 comprises a first jaw 202 (also known as a “cartidge jaw” or a “channel”) and a second jaw 204 (also known as an “anvil jaw” or just “anvil”) movable relative to the cartridge jaw 202 between an open position and a closed position. The cartridge jaw 202 and anvil 204 may be elongated in form. The cartridge jaw 202 defines an elongated channel 208 for receiving a staple cartridge 210 (also known as a “reload”). The anvil 204 has a proximal end 204A, a distal end 204B, and a ramp surface 216 defined at the proximal end 204A, which is described in greater detail below with respect to FIGS. 4 and 9A-9D. The cartridge jaw 202 and anvil 204 are pivotally coupled via a pivot pin 212 that extends through the cartridge jaw 202 and the anvil 204. As seen in FIG. 7, one or more biasing springs 214 extend between the cartridge jaw 202 and anvil 204 to bias the anvil 204 to the open position.

The ramp surface 216 may be visible via a kidney bean-shaped opening 222 (which may be formed as part of the manufacturing process to make the ramp surface 216) that has a first lateral end 222A and a second lateral end 222B. In other words, the kidney bean-shaped opening may be open at its lateral ends 222A, 222B (FIG. 3). As seen in FIG. 4 the ramp surface 216 forms a lower surface of the kidney bean-shaped opening 222. The ramp surface 216 can be arcuately shaped. For example, as shown particularly in FIGS. 4 and 9A-9D, it may be upwardly sloped at a first angle 218 and arcuately taper, in a distal direction, to a substantially horizontal second angle 220.

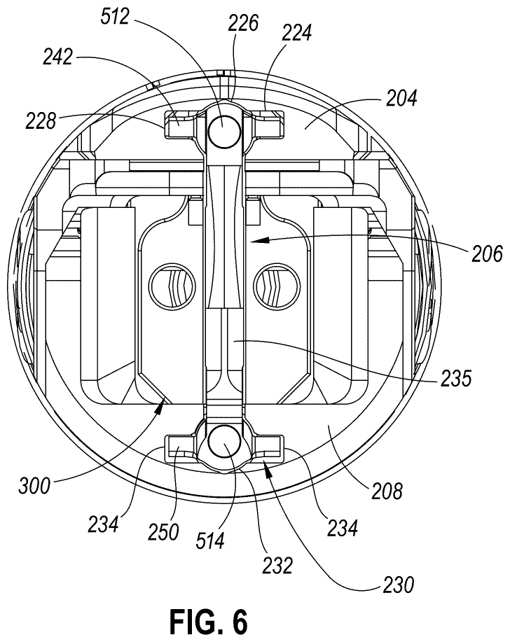

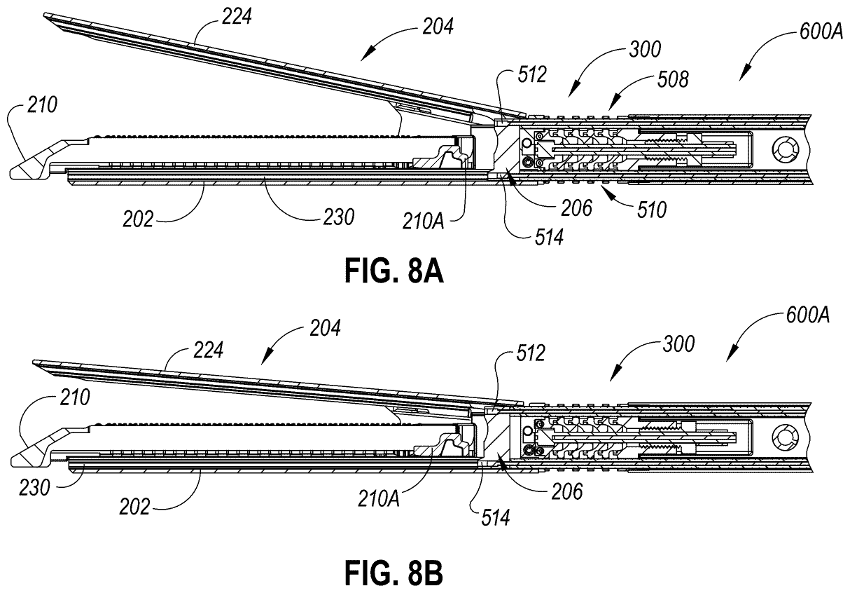

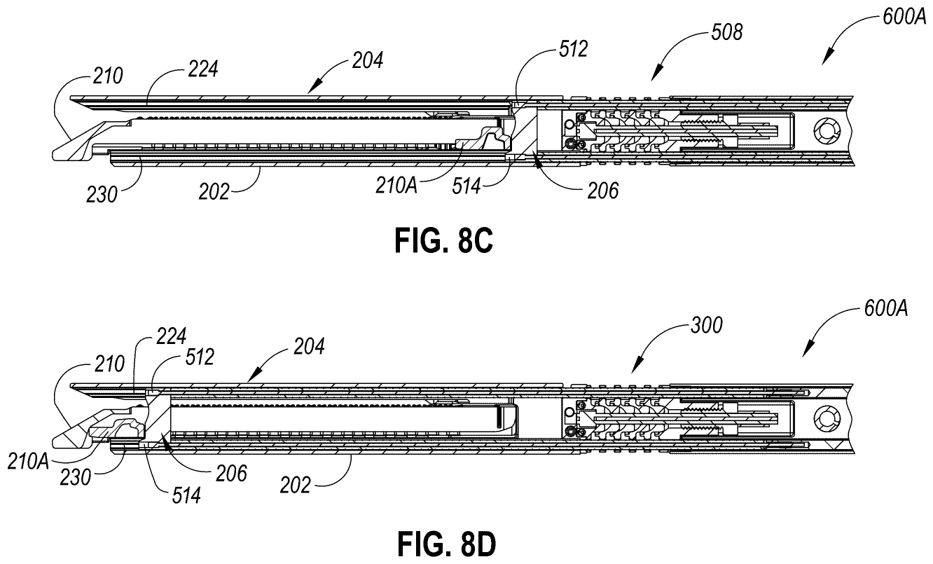

The anvil 204 further defines a longitudinally extending upper knife channel 224 (see FIG. 8A, etc.). As shown particularly in FIG. 6, the upper knife channel 224 includes a centrally disposed cylindrical upper knife channel portion 226 and at least one lateral upper knife channel wing 228 that extends away from the upper knife channel portion 226. While the term ‘cylindrical’ is used, the channel portion 226 need not resemble a perfect cylinder.

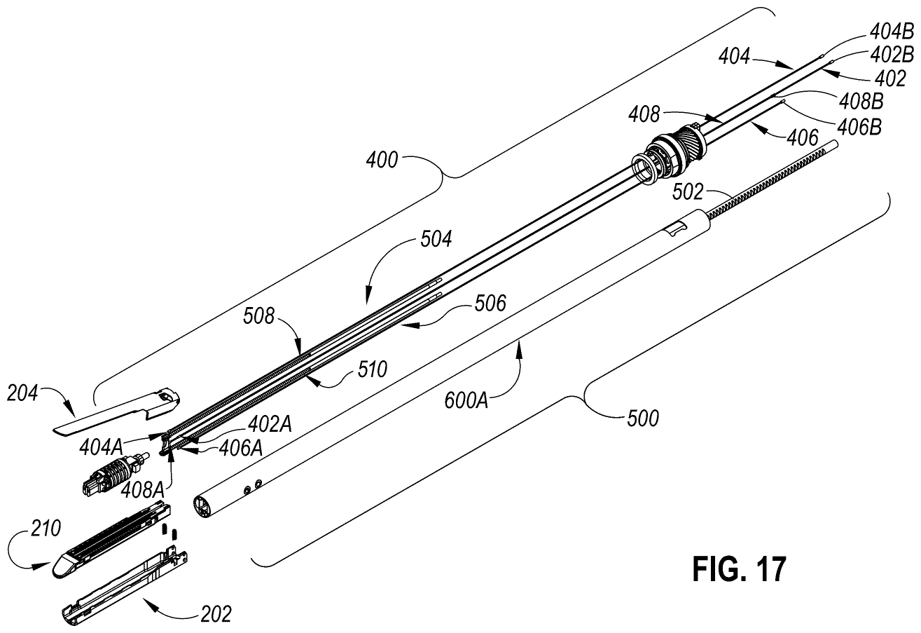

As shown in FIGS. 2 and 17, the surgical instrument 1000 further comprises a knife firing subsystem 500 operable to close the anvil 204 during a closure stroke. After the end effector 200 is closed, the knife firing subsystem 500 is operable to incise and staple, with staples from the staple cartridge 210, the patient tissue captured between the staple cartridge 210 (which is retained by the cartridge jaw 202) and anvil 204 during a firing stroke.

As shown best in FIGS. 4-6, the knife firing subsystem 500, explained further below in greater detail, includes a knife 206 having a knife sled 236. The knife sled 236 is the non-cutting element of the knife 206 and functions as a firing driver by driving cartridge sled 210A distally through a firing stroke, as described below. In some instances, knife sled 236 may be referred to as an I-beam. The knife sled 236 includes an upper knife tab 238, a lower knife tab 246, and a vertical column 235 coupling and extending between upper knife tab 238 and lower knife tab 246. The upper knife tab 238 includes a centrally disposed cylindrical upper knife tab portion 240 and at least one upper knife tab lateral wing 242 that extends away from the upper knife tab portion 240. While the term ‘cylindrical’ is used, the tab portion need not resemble a perfect cylinder.

The upper knife tab 238 may include a pair of lateral wings 242 configured to slidably ride in the upper knife channel 224 to move the anvil 204 between the open position, the grasping position, and the clamping position. Accordingly, the end effector 200 employs “knife-based closure” in which closure of the anvil 204 relative to the channel 208 is driven by distal advancement of the knife 206. Each lateral wing 242 may include a ramped surface 242A that engages the anvil ramp surface 216. The upper knife tab portion 240 defines an upper knife tab opening 244 that is configured to receive a barrel crimp coupled to a center cable 512, which is described in greater detail below. The lower knife tab 246 includes a centrally disposed cylindrical lower knife tab portion 248 and at least one lower knife tab lateral wing 250 that extends away from the lower knife tab portion 248. While the term ‘cylindrical’ is used, the lower knife tab portion 248 need not resemble a perfect cylinder. In some versions, the lower knife tab 246 includes a pair of lateral wings 250. The lower knife tab portion 248 defines a lower knife tab opening 252 that is configured to receive a barrel crimp coupled to a center cable 514, as described in greater detail below.

The staple cartridge 210 may be generally constructed and operable in accordance with the teachings of U.S. patent application Ser. No. 18/588,684, entitled “Methods of Surgical Stapling,” filed on Feb. 27, 2024, the disclosure of which is incorporated by reference herein in its entirety. In use, the end effector 200 is positioned relative to patient tissue such that the staple cartridge 210 is disposed on a first side of the tissue and the anvil 204 is positioned on an opposed second side of the tissue. The anvil 204 is then approximated toward the staple cartridge 210 to compress and clamp the tissue against the deck of the staple cartridge 210. Thereafter, the surgical instrument 1000 is fired so that the knife 206 advances distally through the staple cartridge 210 to both cut the clamped tissue and simultaneously actuate staple drivers housed within the staple cartridge 210 to drive an array of staples into the clamped tissue on either side of the cut line. Staple cartridge 210 defines an elongate knife channel 215 dimensioned to receive a portion of vertical column 235 in order to accommodate advancement of knife 206 through staple cartridge 210. A portion of cartridge sled 210A is slidably housed within elongate knife channel 215 such that vertical column 235 drives cartridge sled 210A distally as knife 206 advances distally in accordance with the description herein (see FIGS. 8C-8D). In some instances, cartridge sled 210A remains in the distal position (see FIG. 8D) relative to the rest of staple cartridge 210, even after knife 206 is retracted proximally after firing staple cartridge 210 in accordance with the description herein.

As mentioned above, cartridge jaw 202 defines an elongated channel 208 for receiving staple cartridge 210. Additionally, cartridge jaw 202 also defines a lower knife channel 230 (see FIGS. 4, 6, and 8A-9D) dimensioned to slidably receive lower knife tab 246. Referring to FIG. 6, the lower knife channel 230 includes a centrally disposed cylindrical lower knife channel portion 232 and at least one lateral lower knife channel wing 234 that extends away from the lower knife channel portion 232. Cylindrical lower knife channel portion 232 is in communication with elongated channel 208 such that when staple cartridge 210 is suitably coupled to cartridge jaw 202, elongate knife channel 215 of staple cartridge 210 and centrally disposed cylindrical lower knife channel portion 232 are aligned to accommodate actuation of knife sled 236 within both channels 215, 230. Lateral lower knife channel wings 234 are dimensioned to slidably house a respective lower knife tab lateral wing 250. Lower knife tab lateral wings 250 are configured to slidably contact lateral lower knife channel wings 234 as knife 206 is advanced in accordance with the description herein. Contact between lower knife tab lateral wings 250 and lateral lower knife channel wings 234 cooperatively assists lateral wings 242 and upper knife channel 224 to close anvil 204 relative to channel 208 in accordance with the description herein. While the term ‘cylindrical’ is used, the channel portion 232 need not resemble a perfect cylinder. Other arrangements of staple cavities and staples may be possible. For example, in some versions, a lower knife channel 230 can be defined in the cartridge jaw 202.

Further to the above, the knife sled 236 is moved distally and proximally by a firing rod 502. The firing rod 502 is configured to apply an indirect force to the knife sled 236, via push coils 508, 510 that directly engage the knife sled 236 (discussed in greater detail below), and push the knife sled 236 toward the distal end of the end effector 200 through a firing stroke. As the firing rod 502 is advanced distally, knife sled 236 rides in the lower knife channel 230 and the upper knife channel 224. At the onset of travel, the upper knife tab 238 rides along the anvil ramp surface 216. Specifically, as particularly seen in the sequence of FIGS. 8A-8D and 9A-9D, movement of the knife sled 236 distally causes the upper knife tab ramped surface 242A to slide along the anvil ramp surface 216. This movement first urges the anvil 204 closed to a position (e.g., FIGS. 8B and 9B) where a compressive force is applied to the tissue sufficient to grasp it (referred to as the grasping position). Continued movement of the knife sled 236 up the ramp surface 216 (e.g., see FIGS. 8C and 9C) results in a compressive force being applied to the tissue (referred to as the clamping position). As the anvil ramp surface 216 transitions to its substantially horizontally angled surface 220 (e.g., see FIGS. 8D and 9D), the upper knife tab 238 can slide within the upper knife channel 224 to drive the stapling and transection of the tissue.

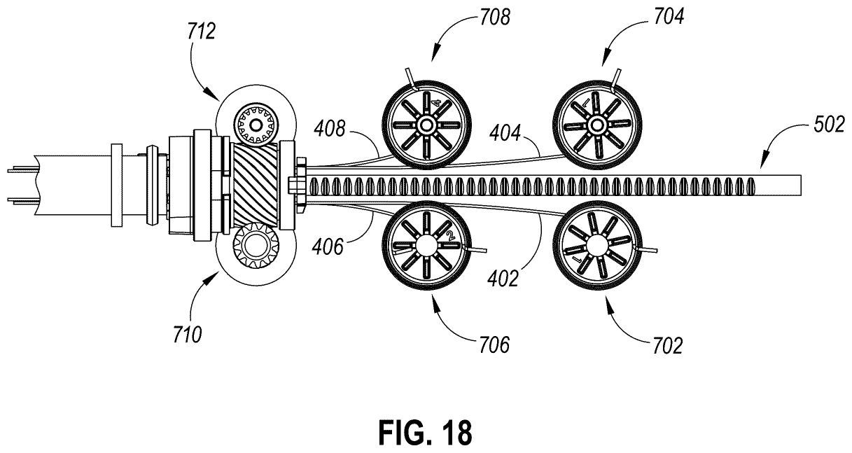

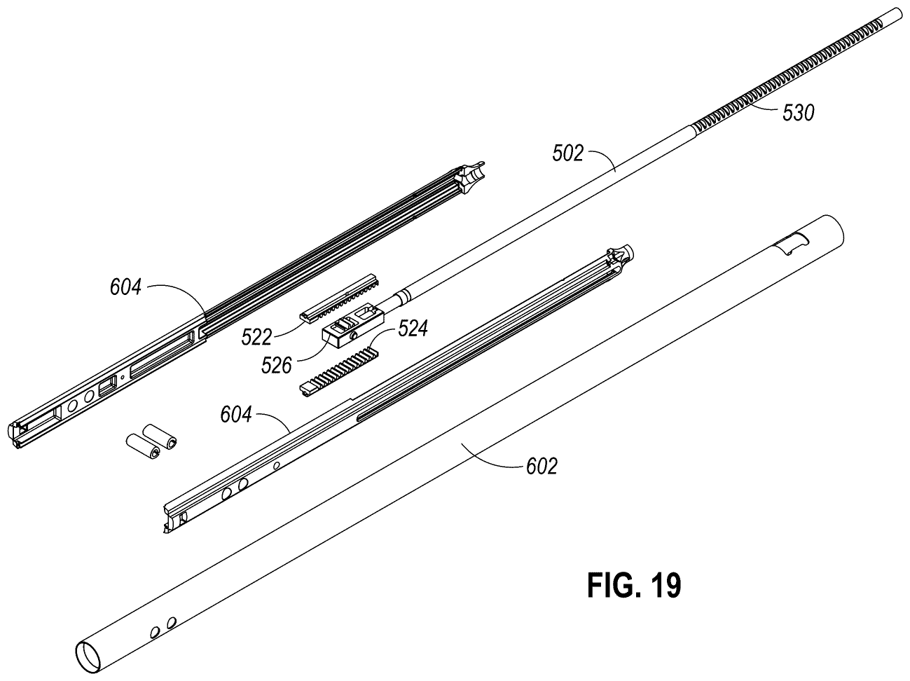

As shown in FIG. 1, the surgical instrument 1000 further comprises a body exemplified as a housing 700 configured to engage a robotic platform (not shown). In other versions, the body may be configured as a handle configured to be gripped and manipulated by a clinician. As best shown in FIGS. 1 and 19, a shaft assembly 600A extends distally from the housing 700 and includes a rotatable outer shaft 602 and an inner shaft 604 arranged in two clamshell halves, with the outer shaft 602 being rotatably mounted to the housing 700 about a rotation joint (not shown), which may include one or more bearings. The inner shaft 604 is rotationally fixed to the outer shaft 602 and is configured such that articulation cables 402, 404, 406, 408 can be partially wound therearound without becoming tangled. As shown in FIG. 18, the housing 700 may house (1) a firing puck assembly 712 as part of the knife firing subsystem 500 operable to close the end effector 200, fire staples, and transect tissue, (2) a set of articulation puck assemblies 702, 704, 706, 708 as part of the articulation subsystem 400 operable to articulate the end effector 200 relative to the shaft assembly 600A, and (3) a shaft roll puck assembly 710 as part of the roll subsystem 600 configured to roll the outer shaft 602.

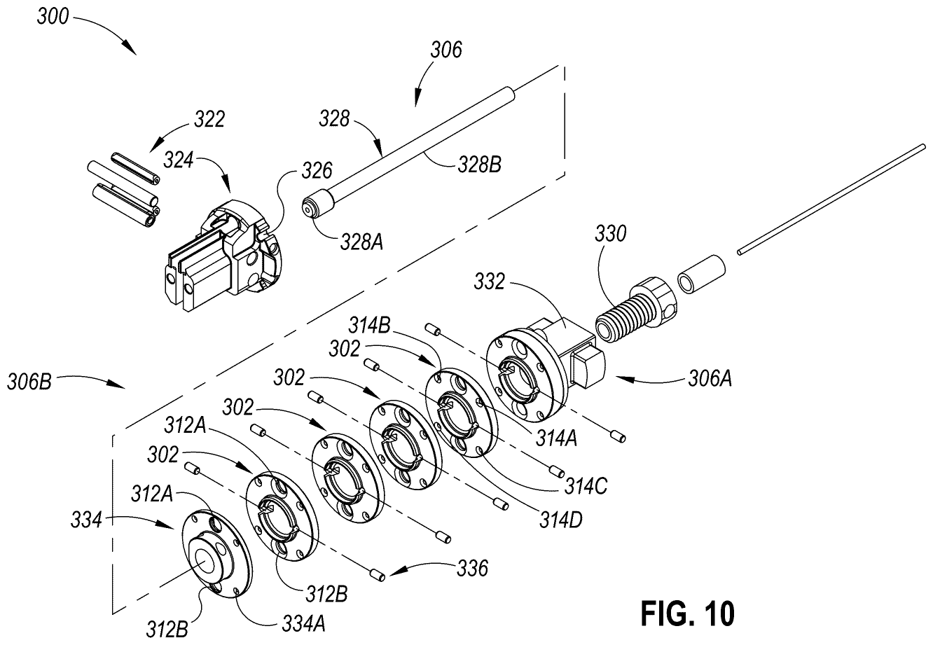

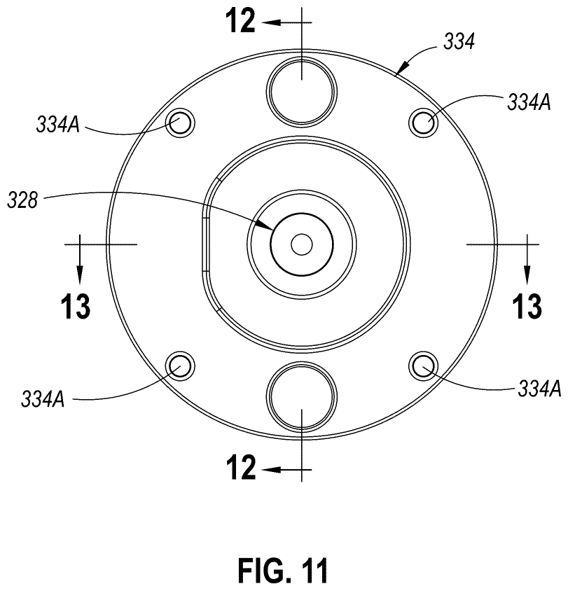

Referring to FIGS. 10-13, the articulation joint 300 comprises an array of joint discs 302 arranged longitudinally, and a center beam assembly 306 that cooperates with the joint discs 302 to provide articulation of the end effector 200 with at least two degrees of freedom (e.g., yaw and pitch), as described further below. Each joint disc 302 includes a central opening 304 that is configured to align coaxially with the central opening 304 of the other joint discs when the articulation joint 300 is in a straight, non-articulated state. The center beam assembly 306 extends longitudinally through the central openings 304 of joint discs 302 and applies a compressive axial force to the array of joints discs 302 to couple the joint discs 302 with one another. The joint discs 302 are nestably stacked with one another along the center beam assembly 306 such that longitudinally adjacent joint discs 302 movably interface with one another.

As seen in FIGS. 9A-10, a distal end 306B of the center beam assembly 306 includes a distal retainer 324 that couples the distal end of the articulation joint 300 with a proximal end of the cartridge jaw 202 via one or more fasteners 322, thereby mechanically grounding and retaining the cartridge jaw 202 and thus the end effector 200 relative to the articulation joint 300. The distal retainer 324 includes a plurality of clearance pockets 326 that receive distal ends of articulation cables 402, 404, 406, 408. The distal end 306B further includes a distal retention disc 334 that defines a plurality of cable retention openings 334A. A proximal end 306A of the center beam assembly 306 includes a proximal retainer 332 that couples the proximal end of the articulation joint 300 with a distal end of the shaft assembly 600A.

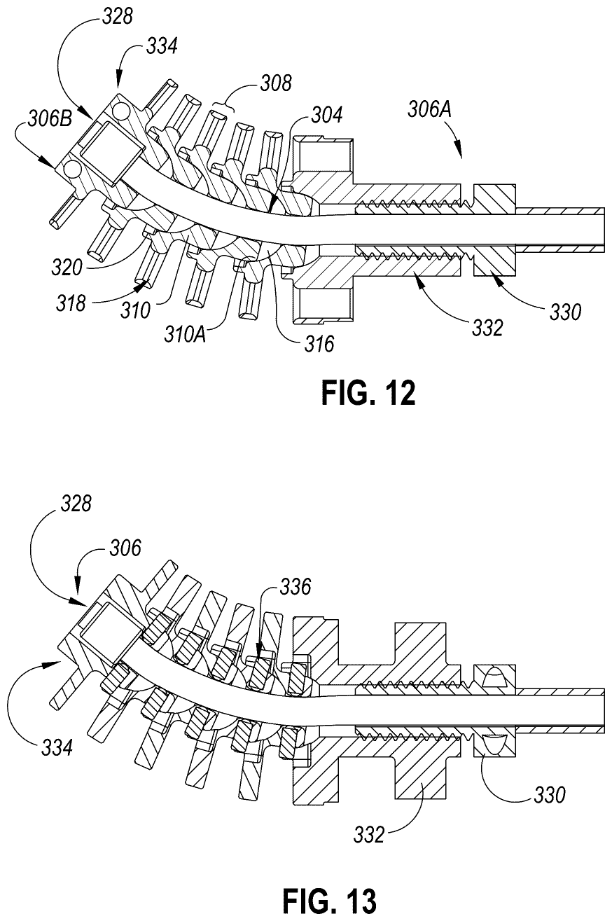

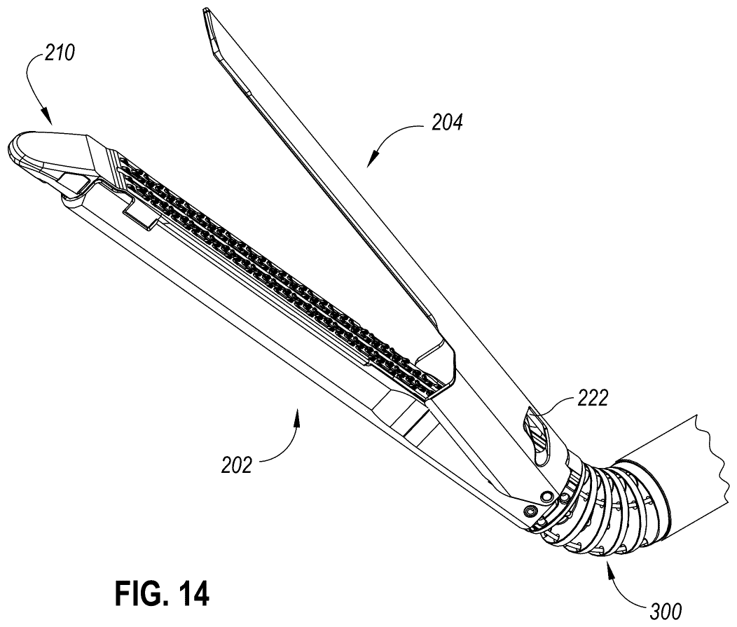

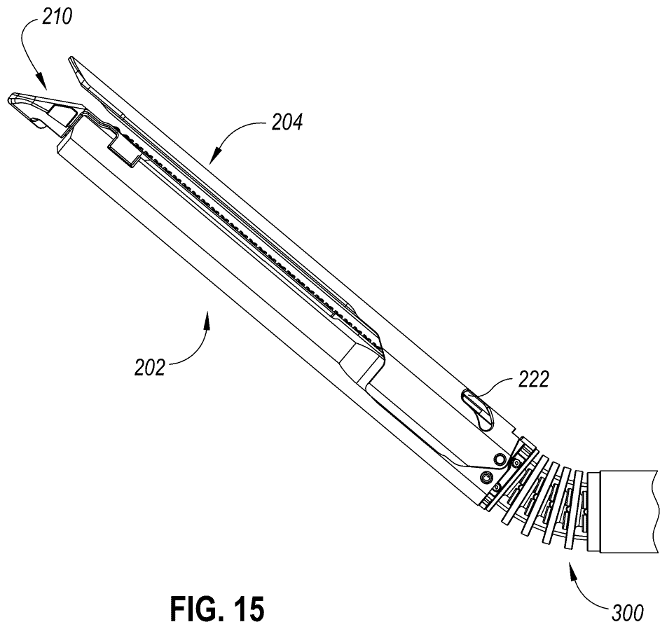

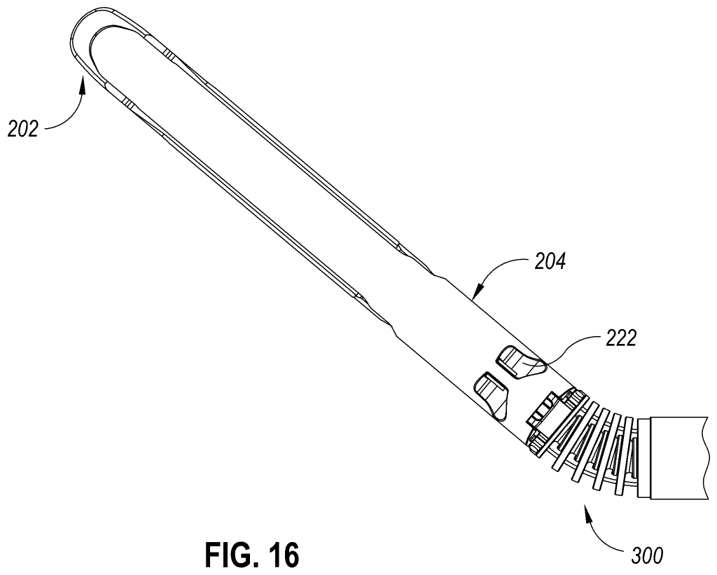

As shown particularly in FIGS. 10, 12, and 13, each joint disc 302 includes an articulation socket 308, an articulation pin 310 protruding outwardly from the articulation socket 308, a first push coil opening 312A defined through the articulation socket 308 and configured to receive a first push coil 508 therethrough, a second push coil opening 312B defined through the articulation socket 308 and configured to receive a second push coil 510 therethrough, and a plurality of articulation cable openings 314A-314D (e.g., a first articulation cable opening 314A, a second articulation cable opening 314B, a third articulation cable opening 314C, and a fourth articulation cable opening 314D) defined through the articulation socket 308 and configured to receive a respective articulation cable 402, 404, 406, 408 (e.g., a first articulation cable 402, a second articulation cable 404, a third articulation cable 406, and a fourth articulation cable 408) therethrough, and discussed in greater detail below. As shown in FIGS. 12 and 13, the central opening 304 is defined in the articulation pin 310 of each joint disc 302. In some versions, three articulation cable openings 314A, 314B, 314C are provided to correspond to three articulation cables 402, 404, 406, while in other versions, four articulation cable openings 314A, 314B, 314C, 314D are provided to correspond to four articulation cables 402, 404, 406, 408.

Each joint disc 302 further includes a rounded articulation pin proximal end 310A and a semi-spherical pin-receiving opening 316 defined in the articulation socket 308. As shown particularly in FIGS. 12 and 13, each rounded articulation pin proximal end 310A pivotally engages in an adjacent pin-receiving opening 316 of an adjacent joint disc 302, with the exception of a proximal-most end 310A that engages with the proximal retainer 332. The articulation pin proximal end 310A and pin-receiving opening 316 interface functions in a similar manner as a swivel bearing. Moreover, the articulation socket 308 includes a socket disc 318 and a pin retention socket 320. A pair of pins 336 are used to provide rotational coupling about a primary axis of the shaft assembly 600A from one disc 302 to the next. In other words, the pins constrain a rotational degree of freedom between adjacent joint discs 302 about the roll axis RA of the instrument 1000. In alternative versions, this feature can be integral to the joint disc 302.

The center beam assembly 306 further includes a center beam 328 that extends longitudinally through the central openings 304 of the joint discs 302. The center beam 328 includes a nitinol core 328A and a stainless-steel collar 328B wound over the nitinol core 328A that allows the center beam 328 to resiliently flex during deflection of the articulation joint 300. The wound stainless-steel collar 328B may have clockwise braiding and counterclockwise braiding to prevent unwinding thereof. The center beam assembly further includes a jack screw 330 that is threadably coupled with the proximal retainer 332 to adjust an axial compression force exerted by the center beam 328 on the array of joint discs 302, thereby enabling adjustment of a pre-load of the articulation joint 300.

The above-described articulation joint 300 forms a portion of the cable articulation subsystem 400 which allows for precise 360-degree movement of the end effector 200 about the articulation joint 300 with at least two degrees of freedom. In some versions, and as dictated by the roll subsystem 600 as well as a need to limit the amount of wrap of the articulation cables 402, 404, 406, 408, the articulation joint 300 is permitted about 320 degrees of roll within the overall system. The cable articulation subsystem 400 also includes a plurality of articulation cables 402, 404, 406, 408 each having a distal end 402A, 404A, 406A, 408A, coupled to the distal end 306B of the center beam assembly 306, and a proximal end 402B, 404B, 406B, 408B. More specifically, each distal end 402A, 404A, 406A, 408A can include a crimp that engages a cable retention opening 334A of the distal retention disc 334 to maintain its positioning. Each articulation cable is discretely manipulable to cause rotation of the articulation joint 300 and end effector 200 about at least one of a pitch axis PA and a yaw axis YA.

In some versions, three articulation cables may be provided rather than the four cables 402, 404, 406, 408 depicted herein. However, four articulation cables 402, 404, 406, 408 circumferentially spaced approximately ninety degrees from one another (as shown) provide load splitting. Additionally, in alternative versions, three and fourth articulation cable configurations may be spaced non-symmetrically relative to one another.

The shaft assembly 600A and housing 700 also form portions of the cable articulation subsystem 400. More specifically, each articulation cable 402, 404, 406, 408 extends from the articulation joint 300 and through the shaft assembly 600A to the housing 700. The proximal end 402B, 404B, 406B, 408B of each articulation cable (402, 404, 406) is movably mounted in the housing 700 which causes the above-mentioned rotation of the articulation joint 300 and end effector 200. The housing 700 includes articulation puck assemblies 702, 704, 706, 708 with rotatable capstans (not shown) about which corresponding proximal ends 402B, 404B, 406B, 408B of the articulation cables 402, 404, 406, 408 are windably mounted.

The articulation cables 402, 404, 406, 408 are routed through the shaft assembly 600A such that they are disposed between the outer shaft 602 and the inner shaft 604, with the articulation cables 402, 404, 406, 408 being able to partially wind therearound without becoming tangled. The inner shaft 604 also prevents the articulation cables 402, 404, 406, 408 from interfering with other components running down the center of the instrument 1000 (through the inner shaft 604).

The articulation cables 402, 404, 406, 408 are routed and coupled to the end effector 200 via the articulation joint 300 such that movement thereof in a proximal direction (via winding about the capstans of the housing 700) causes the end effector 200 to articulate in a predetermined manner via the articulation joint 300. For example, actuation of the first articulation cable 402 in the proximal direction causes articulation of the end effector 200 upwards and to the left, actuation of the second articulation cable 404 in the proximal direction causes rotation of the end effector 200 upwards and to the right, actuation of the third articulation cable 406 in the proximal direction causes rotation of the end effector 200 downwards and to the left, and actuation of the fourth articulation cable 408 in the proximal direction causes rotation of the end effector 200 downwards and to the right. Similarly, movement of two articulation cables simultaneously will result in blended articulation of the end effector 200. As will be appreciated by those skilled in the art, this configuration provides for the above-mentioned precise 360-degree articulation of the end effector 200 via the articulation joint 300 with at least two degrees of freedom and about 320 degrees of roll.

As shown throughout FIGS. 2, 4, 5, 8A-8D, 9A-9D, 17 and 19, the knife firing subsystem 500 includes the aforementioned knife 206, the aforementioned knife sled 236, a firing rod 502 that drives the knife 206 and/or knife sled 236, a first push rod 504, and a second push rod 506. The firing rod 502 includes a firing rod rack 530 and is driven by a firing puck assembly 712 of the housing 700. The first push rod 504 has a first push rod distal end 504A coupled to the knife sled 236 and a first push rod proximal end 504B coupled to the firing rod 502. Similarly, the second push rod has a second push rod distal end 506A coupled to the knife sled 236 and a second push rod proximal end 506B coupled to the firing rod 502. The distal ends 504A, 506A are coupled to respective upper and lower portions of the knife sled 236 (e.g., the upper knife tab 238 and the lower knife tab 246), which enables the knife 206 to be pushed evenly at its ends. In some versions, the proximal ends 504B, 506B of the push rods 504, 506 are coupled to the firing rod 502 via a differential 520.

The knife firing subsystem 500 is configured in a manner to enable articulation of the end effector 200 while still enabling proper functionality of the knife 206. To that end, the first push rod 504 includes a first flexible section in the form of a first push coil 508 and the second push rod 506 comprises a second flexible section in the form of a second push coil 510. The push coils 508, 510 route through the articulation joint 300 via the respective push coil openings 312A, 312B, and the push rods 504, 506 engage the respective tab openings 244, 252 in the knife sled 236. A first center cable 512 extends through the first push coil 508 to engage the knife sled 236 via a barrel crimp, and a second center cable 514 extends through the second push coil 510 to engage the knife sled 236 via a barrel crimp. The push coils 508, 510 provide the push rods 504, 506 sufficient stability to deliver an axial firing force to the knife 206, while not being too stiff that would prevent articulation at the joint 300. The cables 512, 514, which are engaged with the knife sled 236 as discussed above (see, e.g., FIG. 8A), prevent the push coils 508, 510 from stretching and/or elongating and serve as retraction cables when the rods 504, 506 are retracted towards the proximal end of the surgical instrument 1000. The entirety of each push rod 504, 506 does not extend through the articulation joint 300, and therefore does not need to be flexible. Accordingly, a proximal section of each push rod 504, 506 can be less flexible than the push coils 508, 510.

### II. Illustrative Firing Lockouts Coupled with Cartridge Jaw

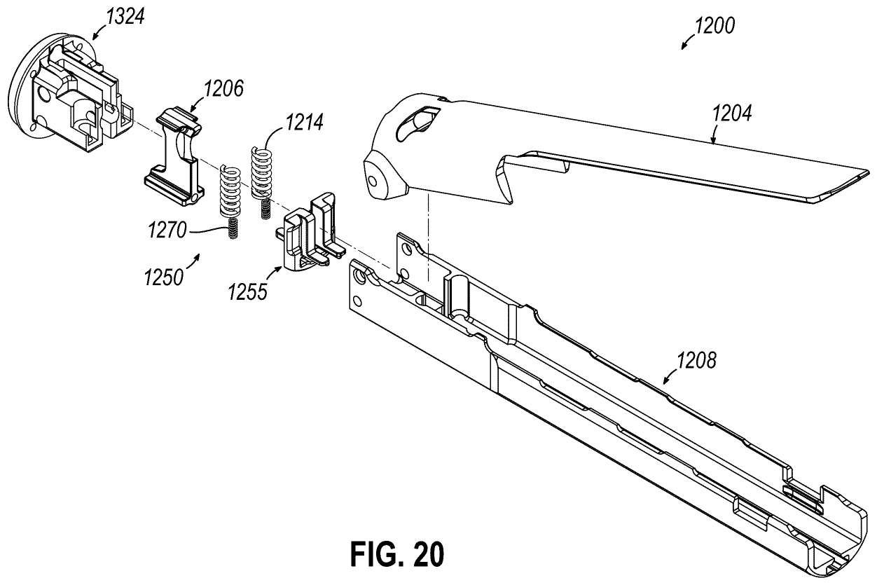

It may be desirable to inhibit firing of the surgical instrument 1000 in instances when the end effector 200 is loaded with a staple cartridge 210 that has already been fired (i.e., a “spent-cartridge” condition), and also when the end effector 200 is not loaded with any staple cartridge at all (i.e., an “absent-cartridge” condition). Attempted firings during such conditions are the result of user error and could result in unintended action on patient tissue; namely, severing the tissue without simultaneously sealing it with staples. The illustrative configurations shown and described below in connection with FIGS. 20-36C are effective to inhibit firing in each of a spent-cartridge condition and an absent-cartridge condition, and thereby protect against such unintended action on patient tissue.

### A. First Illustrative Firing Lockout Assembly

As shown in FIG. 20, end effector 1200 may be substantially similar to end effector 200 except as otherwise shown and described below. End effector 1200 includes, among other features, an anvil 1204, a knife 1206, a lockout assembly 1250, and a distal retainer 1324. End effector 1200 further includes a cartridge jaw that is similar to cartridge jaw 202 and defines an elongate channel 1208 configured to receive a staple cartridge. Lockout assembly 1250 is configured to inhibit firing of end effector 1200 in each of a spent-cartridge condition and an absent-cartridge condition, and permit firing of end effector 1200 only when an unspent staple cartridge is properly positioned (i.e., fully seated) in end effector 1200. Lockout assembly 1250 includes a lockout body 1255 (also referred to herein as a “blocker”), a pair of lockout springs 1270, and pair of biasing springs 1214 that are substantially similar to previously mentioned biasing springs 214. Each biasing spring 1214 may include a flat spring bottom or a washer (as shown) which may rest against a shelf of distal retainer 1324. Flat spring bottom or washer of biasing springs 1214 may thereby act to limit a vertical range of motion of lockout body 1255 relative to the cartridge jaw. As described below, lockout body 1255 is configured to vertically translate within an opening exemplified as a through-opening 1209 formed in the floor of the cartridge jaw.

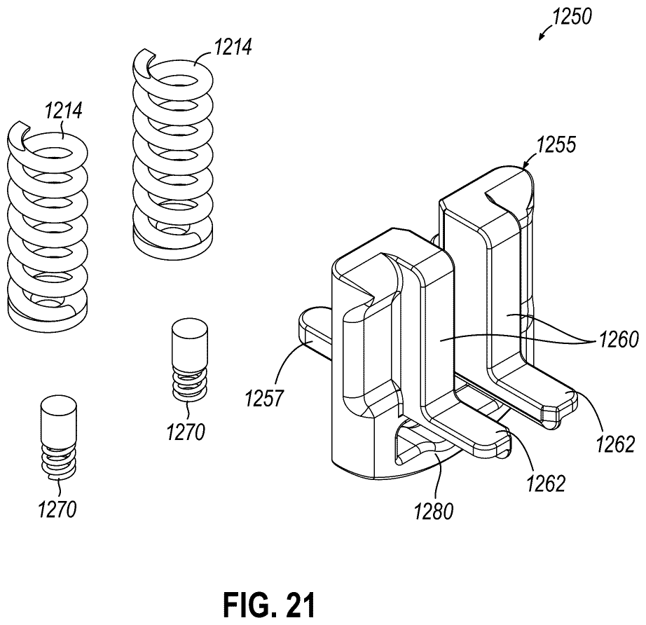

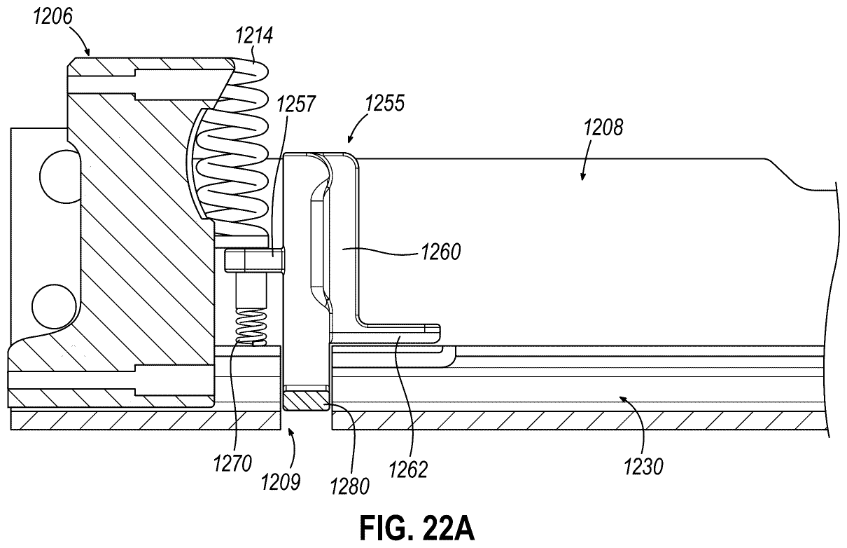

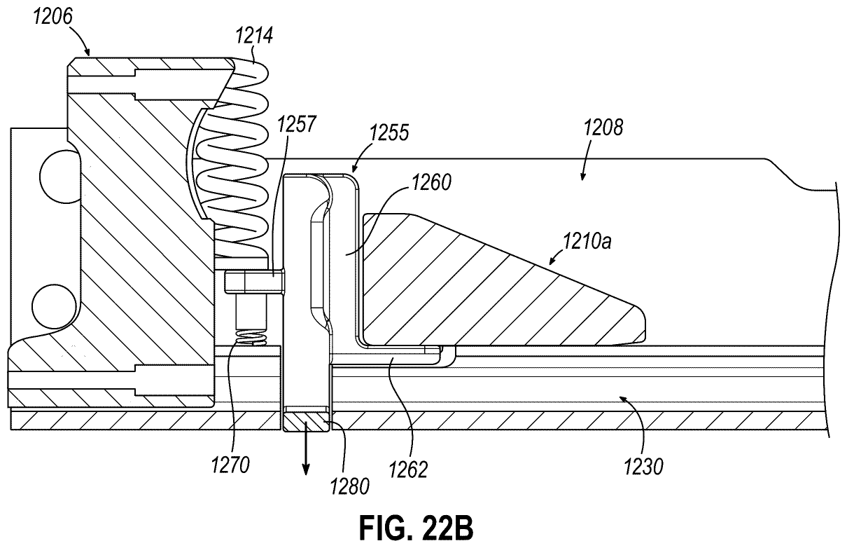

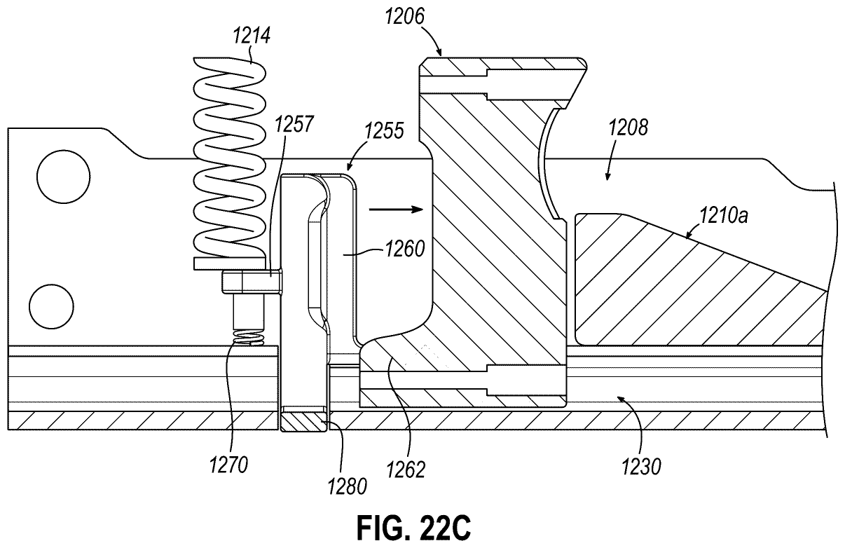

As shown in FIG. 21, lockout body 1255 of lockout assembly 1250 may be generally “U” shaped to include a lockout body base 1280 and a pair of lockout body arms 1260 extending upwardly from laterally opposed portions of base 1280 such that a lateral gap is defined between arms 1260. Each arm 1260 includes a proximally projecting spring protrusion 1257 which each of a respective biasing spring 1214 and lockout spring 1270 may engage. As described below, lockout spring 1270 may bias lockout body 1255 upwards to contact biasing springs 1214 to inhibit (i.e., block) distal advancement of knife 1206 through a staple cartridge. Lockout body 1255 may also include distally projecting lockout body sled protrusions 1262 which can engage sled 1210a as shown in FIG. 22B. Lockout body sled protrusions 1262 are shown as being positioned on lockout body arms 1260 but may optionally be positioned on lockout body base 1280. Lockout body sled protrusions 1262 are shown to include a horizontal engagement surface but may optionally include a ramped surface as described in later lockout assemblies. Biasing springs 1214 and lockout springs 1270 are shown as compression coil springs but may optionally be any suitable alternative capable of applying a biasing force to lockout body 1255.

FIGS. 22A-22C show an illustrative operation of lockout assembly 1250 in end effector 1200. As can be seen in FIG. 22A, with sled 1210a positioned distal to its proximal home position (e.g., when the corresponding staple cartridge, not shown, is in a spent state), lockout spring 1270 biases lockout body 1255 upwardly into a raised position in which lockout body base 1280 is positioned within lower knife channel 1230 to establish a locked configuration in which lockout body base 1280 is positioned to directly contact a distal end of knife 1206 and thus inhibit distal advancement of knife 1206 from its proximal home position.

FIG. 22B shows sled 1210a positioned in a proximal home position when the corresponding staple cartridge in an unspent state is fully seated in end effector 1200. An underside of sled 1210a applies a downward force onto the upwardly facing surfaces of lockout body sled protrusions 1262 to thus drive lockout body 1255 downwards against the biasing force of lockout springs 1270 and into a lowered position in through-opening 1209. This establishes an unlocked configuration of the lockout assembly 1250 in which the knife 1206 is permitted to advance distally between lockout body arms 1260 and over top of lockout body base 1280 such that knife 1206 may contact and drive sled 1210a distally to fire staples on tissue as shown in FIG. 22C. Alternatively, in place of through-opening 1209, the opening may instead be configured as a cavity sized to slidably house lockout body base 1280.

FIG. 22C shows knife 1206 progressed distally and engaged with sled 1210a such that knife 1206 is distally beyond lockout body 1255 and thus free to translate distally through a firing stroke. Once knife 1206 advances distally beyond lockout body 1255, lockout body 1255 automatically returns from the lowered unlocked position to the raised locked position via the bias of lockout springs 1270.

### B. Second Illustrative Firing Lockout Assembly

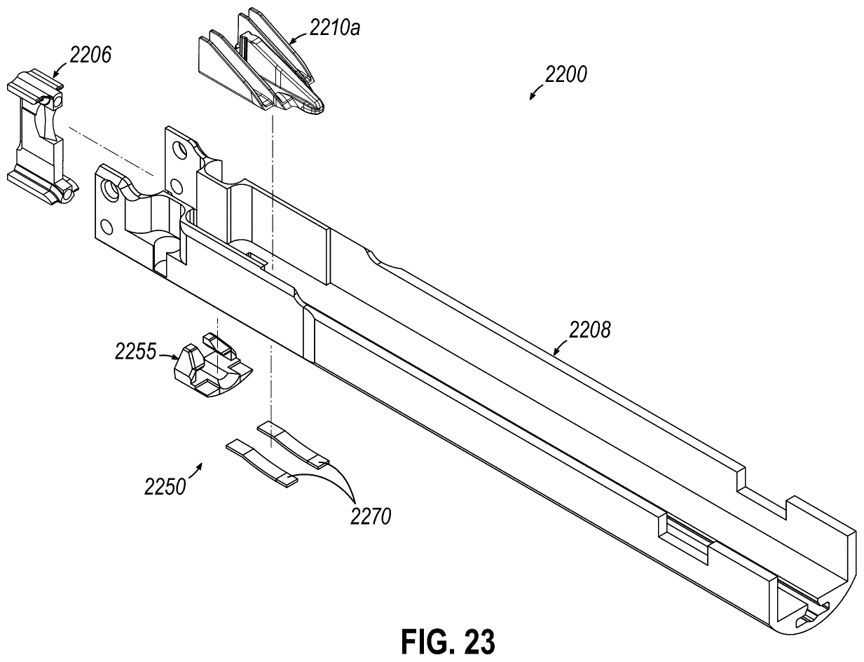

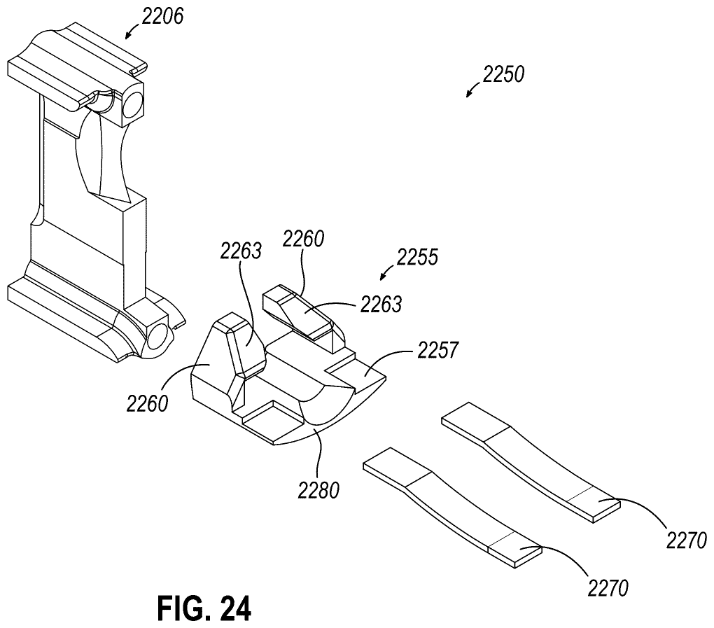

As shown in FIGS. 23-24, end effector 2200 may be substantially similar to end effectors 200, 1200 described above, and in particular is configured to function similar to end effector 1200 except as otherwise shown and described below. End effector 2200 includes, among other features, a knife 2206 and a lockout assembly 2250. End effector 2200 further includes a cartridge jaw that is similar to cartridge jaw 202 and defines an elongate channel 2208 configured to receive a staple cartridge. As described below, lockout assembly 2250 is configured to inhibit firing of end effector 2200 in each of a spent-cartridge condition and an absent-cartridge condition, and permit firing of end effector 2200 only when an unspent staple cartridge is properly positioned (i.e., fully seated) in end effector 2200. Sled 2210a is shown for representative purposes and its use will be described below. As described below, lockout body 2255 is configured to vertically translate within an opening exemplified as a through-opening 2209 formed in the floor of the cartridge jaw.

Lockout assembly 2250 includes a lockout body 2255 (also referred to herein as a “blocker”) and a pair of lockout springs 2270. Lockout body 2255 may be generally “U” shaped and include a lockout body base 2280 and a pair of lockout body arms 2260 extending upwardly from laterally opposed portions of base 2280 such that a lateral gap is defined between arms 2260. Lockout body base 2280 is shown including lockout body spring protrusions 2257 which may alternatively be included on lockout body arms 2260. Lockout body spring protrusions 2257 are coupled with lockout springs 2270 to thus apply a biasing force to lockout body 2255. While lockout assembly 2250 is shown with two lockout springs 2270, it may alternatively include only one lockout spring 2270. Each lockout body arm 2260 may include a lockout body ramp 2263 which slides against an underside of sled 2210a as described below.

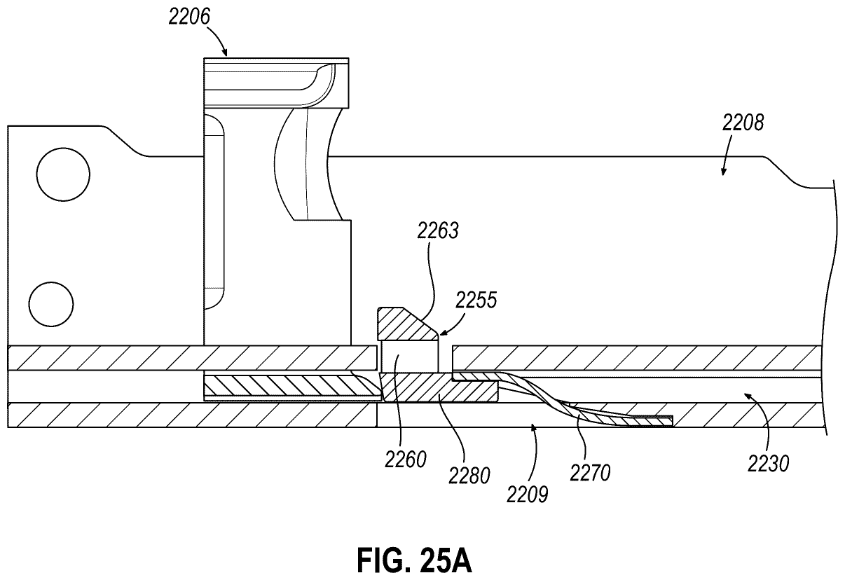

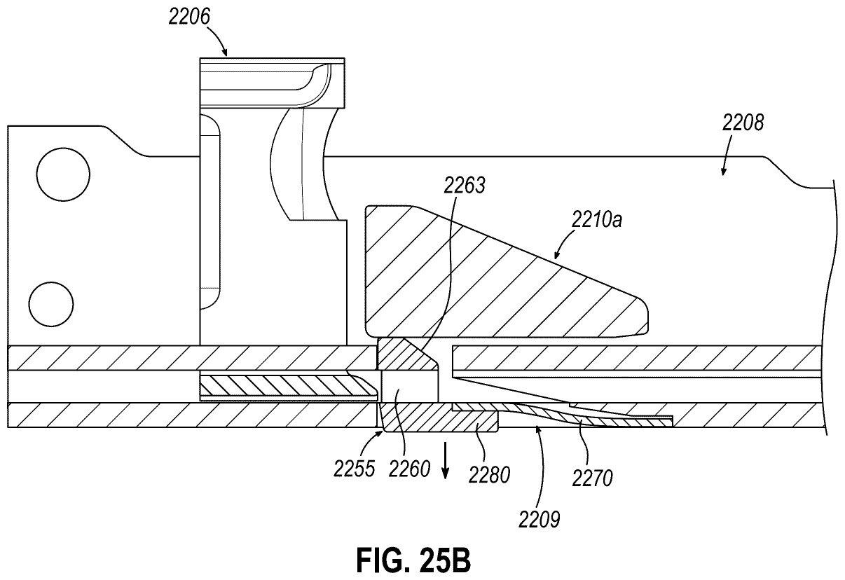

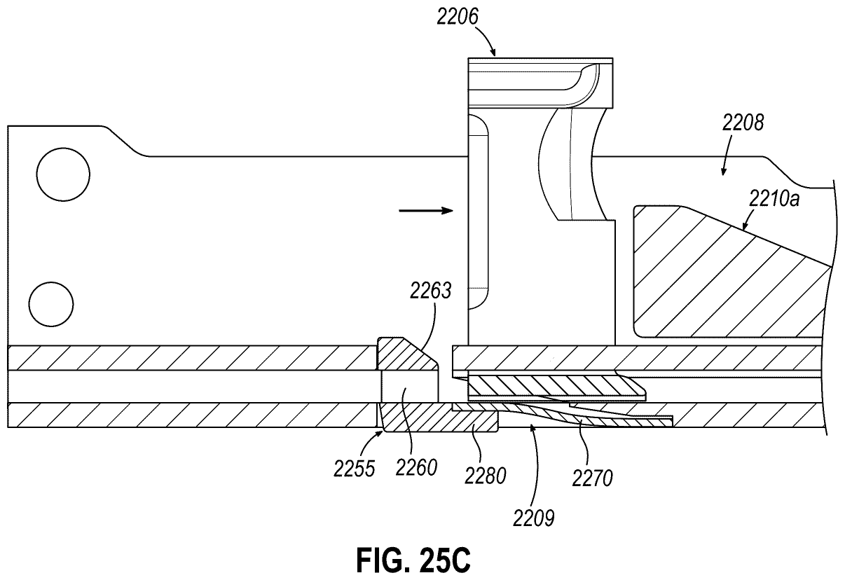

FIGS. 25A-25C show an illustrative operation of lockout assembly 2250. FIG. 25A shows lockout assembly 2250 in a locked configuration with lockout spring 2270 biasing lockout body 2255 upwards into a raised position that defines a locked configuration. While in the locked configuration, lockout body base 2280 is positioned within lower knife channel 2230 to thereby abut a distal end of knife 2206 and thereby inhibit (i.e., block) distal translation of knife 2206.

FIG. 25B shows lockout assembly 2250 after the underside of sled 2210a of an unspent staple cartridge has contacted and driven lockout body 2255 downwards against the biasing force of lockout springs 2270 and into a lowered position in through-opening 2209. This establishes an unlocked configuration of the lockout assembly 2250 in which the knife 2206 is permitted to advance distally between lockout body arms 2260 and over top of lockout body base 2280 such that knife 2206 may contact and drive sled 2210a distally to fire staples on tissue, as shown in FIG. 25C. Alternatively, in place of through-opening 2209, the opening may instead be configured as a cavity sized to slidably house lockout body base 2280.

FIG. 25C shows lockout assembly 2250 still in the unlocked configuration as knife 2206 actuates sled 2210a distally through the staple cartridge along a distal firing stroke. Though not shown, once knife 2206 advances distally fully beyond lockout body 2255, lockout body 2255 automatically returns from the lowered unlocked position to the raised locked position via the bias of lockout springs 2270.

Upon completion of the distal firing stroke, knife 2206 is retracted proximally and a proximal end of knife 2206 engages ramped surface 2263 of lockout body 2255 in the raised position to thereby drive lockout body 2255 downwardly so that knife 2206 may fully return to its proximal home position shown in FIG. 25A. Once knife 2206 is again in the position shown in FIG. 25A, lockout assembly 2250 may again inhibit distal advancement of knife 2206 until an unspent staple cartridge having a proximally positioned sled 2210a is seated within the cartidge jaw as shown in FIG. 25B.

### C. Third Illustrative Firing Lockout Assembly

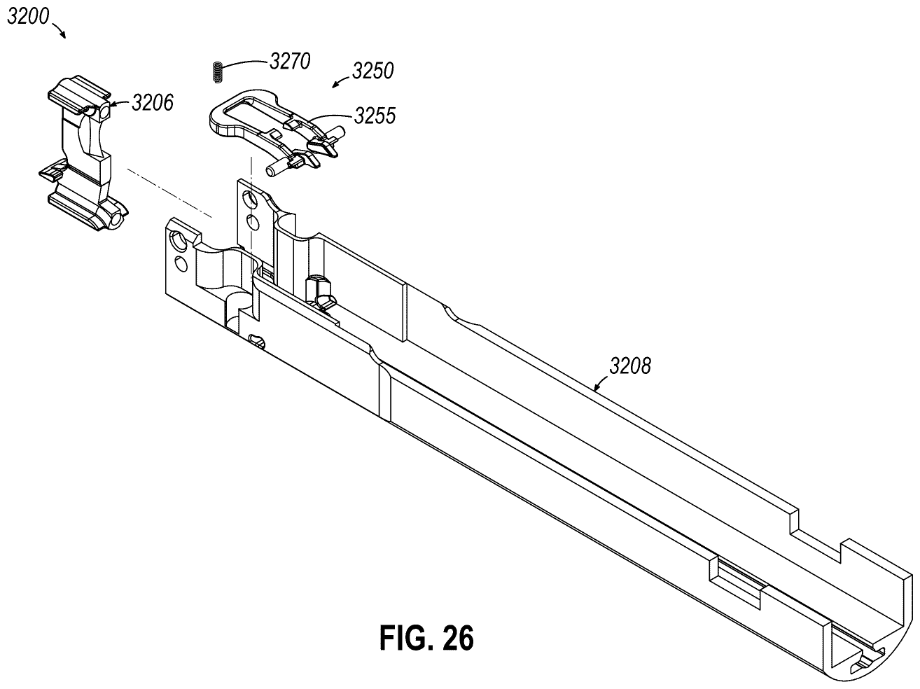

As shown in FIGS. 26-28, end effector 3200 may be substantially similar to end effectors 200, 1200, 2200 described above, except as otherwise shown and described below. End effector 3200 includes, among other features, a knife 3206 and a lockout assembly 3250. End effector 3200 further includes a cartridge jaw that is similar to cartridge jaw 202 and defines an elongate channel 3208 configured to receive a staple cartridge. As described below, lockout assembly 3250 is configured to inhibit firing of end effector 3200 in each of a spent-cartridge condition and an absent-cartridge condition, and permit firing of end effector 3200 only when an unspent staple cartridge is properly positioned (i.e., fully seated) in end effector 3200.

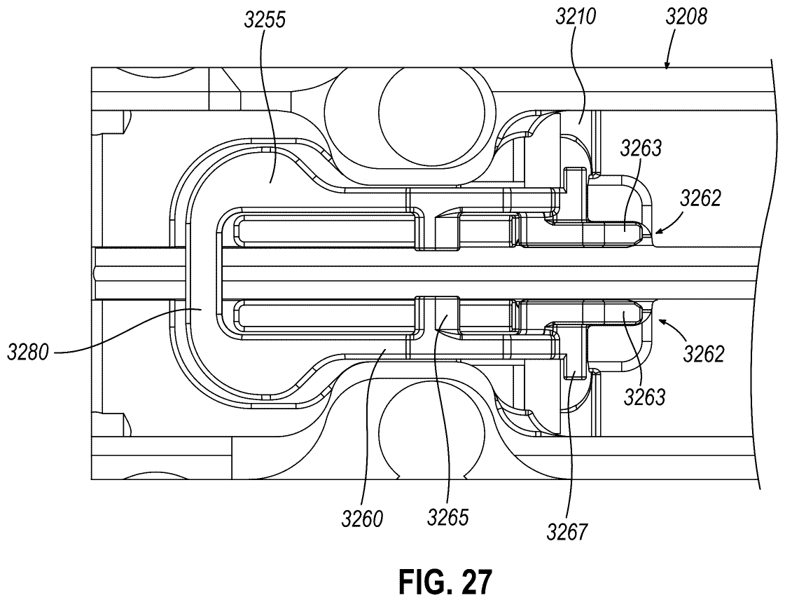

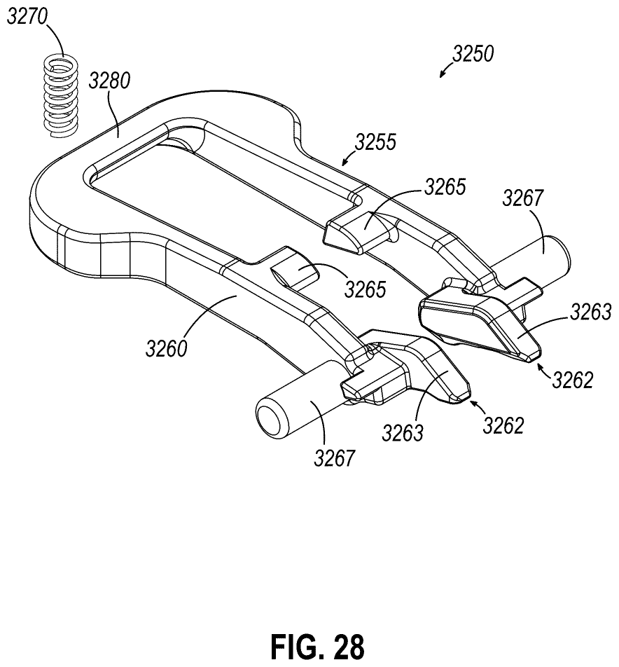

Lockout assembly 3250 includes lockout body 3255 (also referred to herein as a “blocker”) and a lockout spring 3270. FIG. 27 shows a top cross-sectional view of lockout body 3255 positioned inside channel 3208 and with knife 3206 removed. As shown in FIGS. 27-28, lockout body 3255 may be generally “U” shaped and a pair of longitudinally extending lockout body arms 3260 and a cross-bar 3280 that interconnects proximal ends of the lockout body arms 2360. A lockout body pivot post 3267 extends laterally outwardly from a distal end portion of each lockout body arm 3260 and is pivotable within a respective through-opening formed in a corresponding sidewall of the cartridge jaw. Lockout body pivot posts 3267 cooperate to define a laterally extending pivot axis that extends transversely to a longitudinal axis of the cartridge jaw. As described below, this configuration enables lockout body 3255 to pivot relative to the cartidge jaw and knife 3206 between a lowered locked position that inhibits (i.e., blocks) distal advancement of knife 3206 and a raised unlocked position that permits distal advancement of knife 3206.

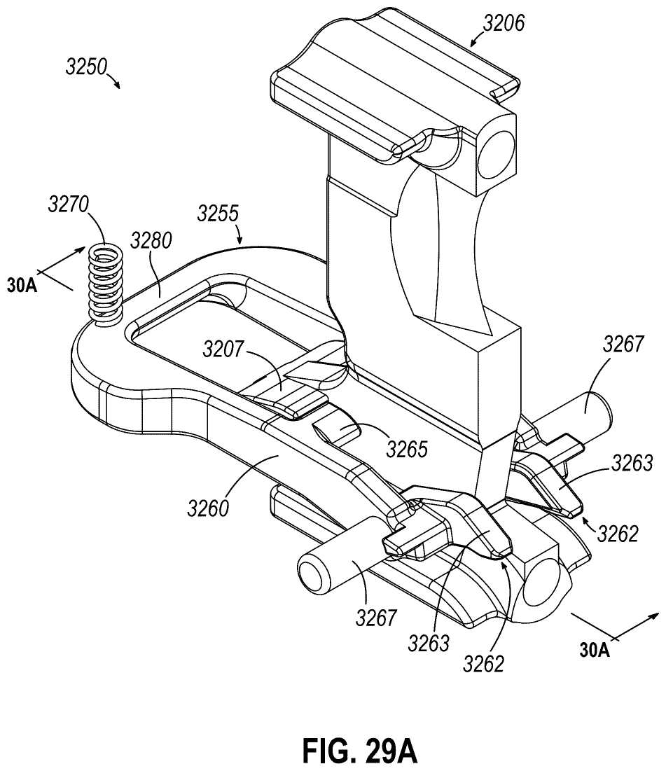

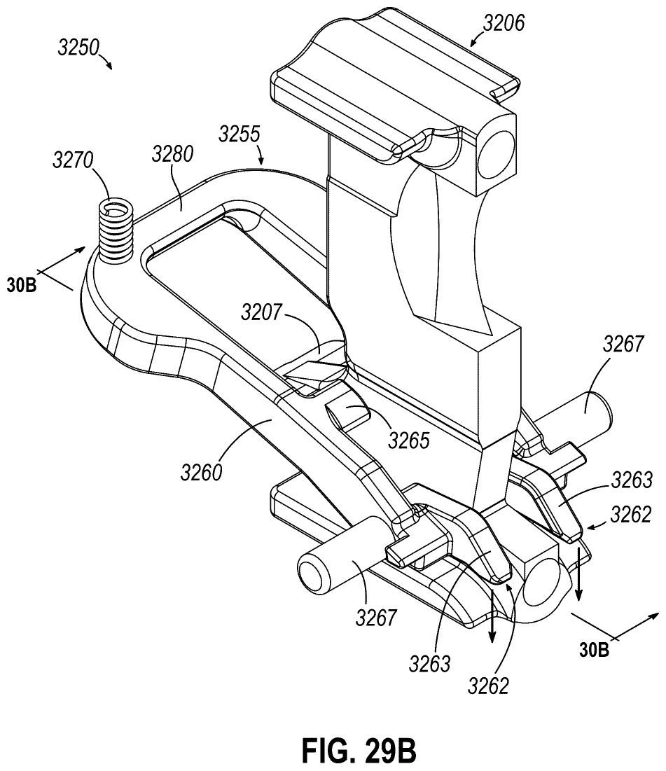

A medial portion of each lockout body arm 3260 includes a laterally inwardly projecting lockout protrusion 3265 configured to directly contact a laterally outwardly projecting side wing 3207 formed on a respective lateral side of knife 3206. A distal terminal end of each lockout body arm 3260 includes a sled engagement protrusion 3262 having a ramped surface 3263 configured to cammingly engage sled 3210a upon insertion of an unspent staple cartridge into the cartridge jaw. While not shown, lockout spring 3270 may be affixed to any portion of end effector 3200 to thus exert a biasing force on lockout body 3255 that biases lockout body 3255 downwardly toward the lowered locked position shown in FIG. 29A.

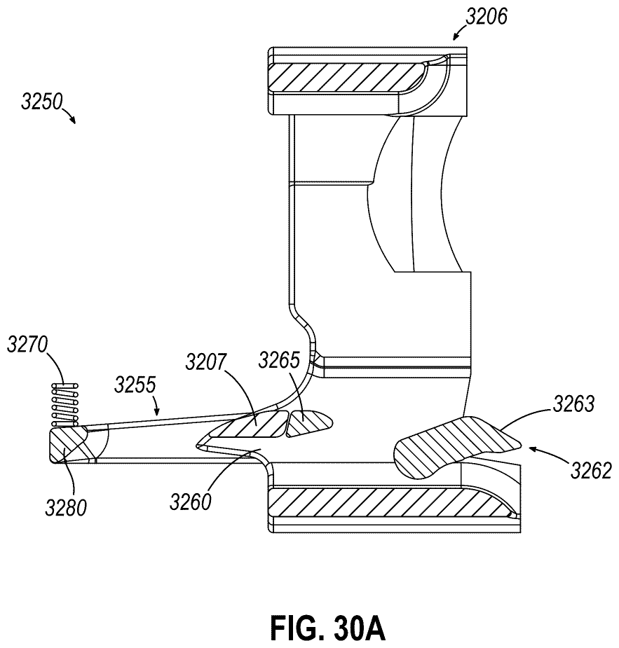

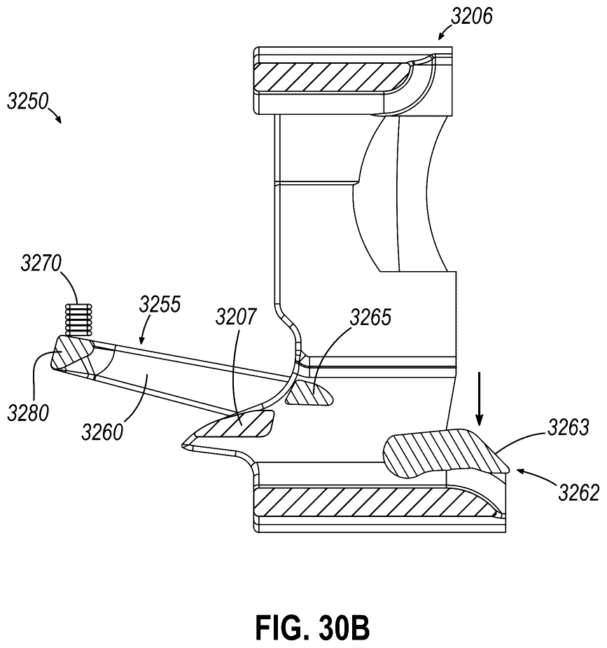

FIGS. 29A-31 show an illustrative operation of lockout assembly 3250. FIGS. 29A and 30A show lockout body 3255 pivoted downwardly in the lowered locked position, toward which lockout body 3255 is biased via a downward force exerted by lockout spring 3270 on lockout body cross-bar 3280. In the lowered locked position of lockout body 3255, a proximally facing surface of each lockout protrusion 3265 is positioned to directly contact a distally facing surface of a corresponding side wing 3207 of knife 3206 and thereby inhibit distal advancement of knife 3206 through end effector 3200.

FIGS. 29B and 30B show lockout body 3255 pivoted upwardly in the raised unlocked position following seating of an unspent staple cartridge into the cartridge jaw. Upon seating of the unspent staple cartridge, an underside of its proximally positioned sled 3210a exerts a downward force on the ramped surfaces 3263 of sled engagement protrusions 3262, thereby driving sled engagement protrusions 3262 downwardly and causing lockout body 3255 to pivot relative to the cartridge jaw via pivot posts 3267 from the lowered position to the raised position and simultaneously compressing lockout spring 3270. In the raised position of lockout body 3255, lockout protrusions 3265 are disengaged from side wings 3207 of knife 3206, thereby permitting distal translation of knife 3206.

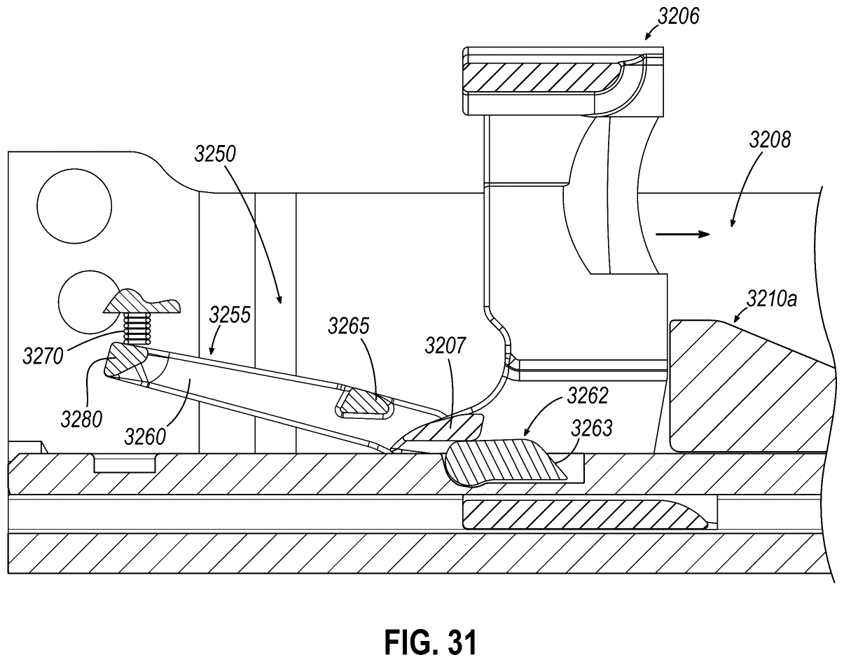

As shown in FIG. 31, knife 3206 may now advance distally through a lateral gap defined between lockout body arms 3260 such that side wings 3207 of knife 3206 pass distally beyond lockout protrusions 3265 and over top of sled engagement protrusions 3262. In this manner, knife 3206 may actuate sled 3210a distally through a firing stroke to deploy staples into tissue compressed by end effector 3200.

### D. Fourth Illustrative Firing Lockout Assembly

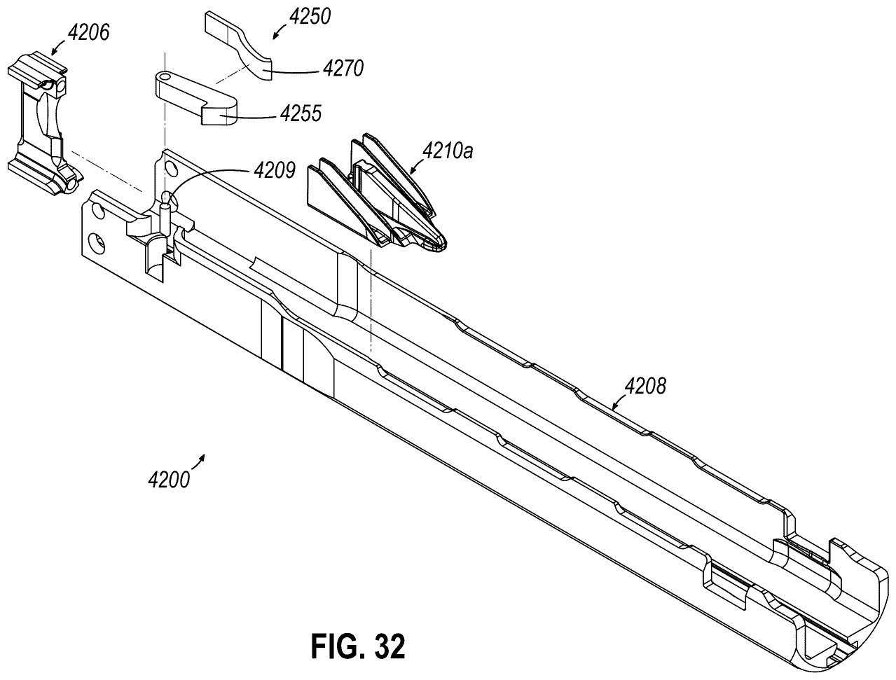

As shown in FIG. 32, end effector 4200 may be substantially similar to end effectors 200, 1200, 2200, 3200 described above, except as otherwise shown and described below. End effector 4200 may include, among other features, a knife 4206, a sled 4210a, and a lockout assembly 4250. End effector 4200 further includes a cartridge jaw that is similar to cartridge jaw 202 and defines an elongate channel 4208 configured to receive a staple cartridge. As described below, lockout assembly 4250 is configured to inhibit firing of end effector 4200 in each of a spent-cartridge condition and an absent-cartridge condition, and permit firing of end effector 4200 only when an unspent staple cartridge is properly positioned (i.e., fully seated) in end effector 4200.

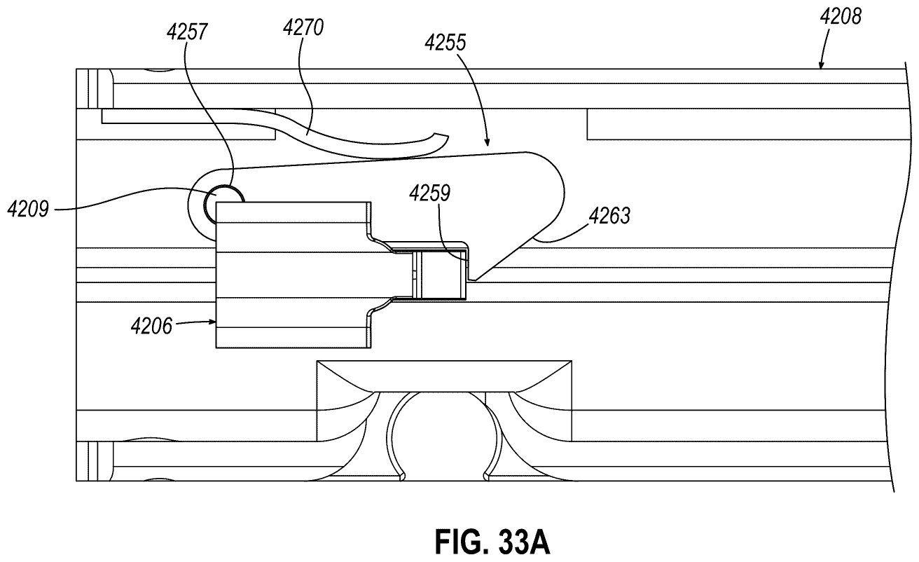

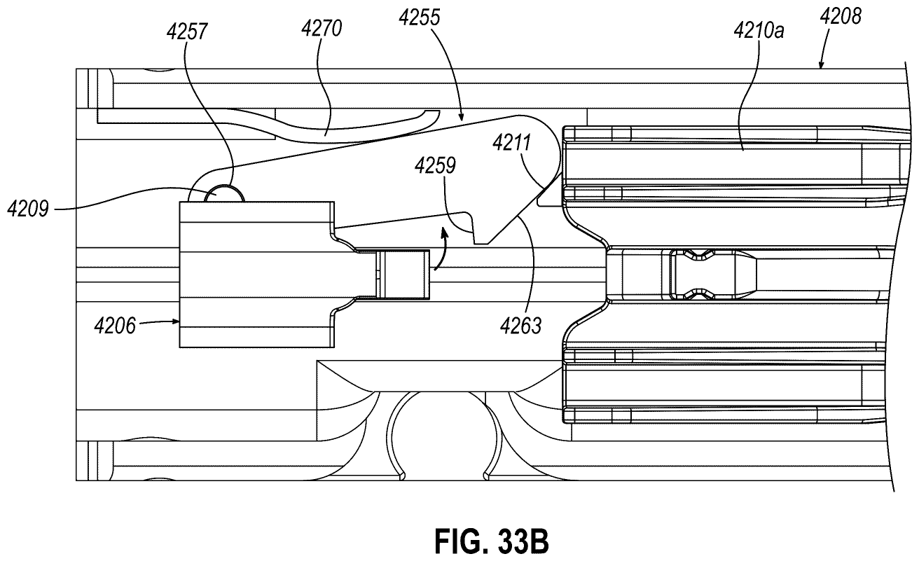

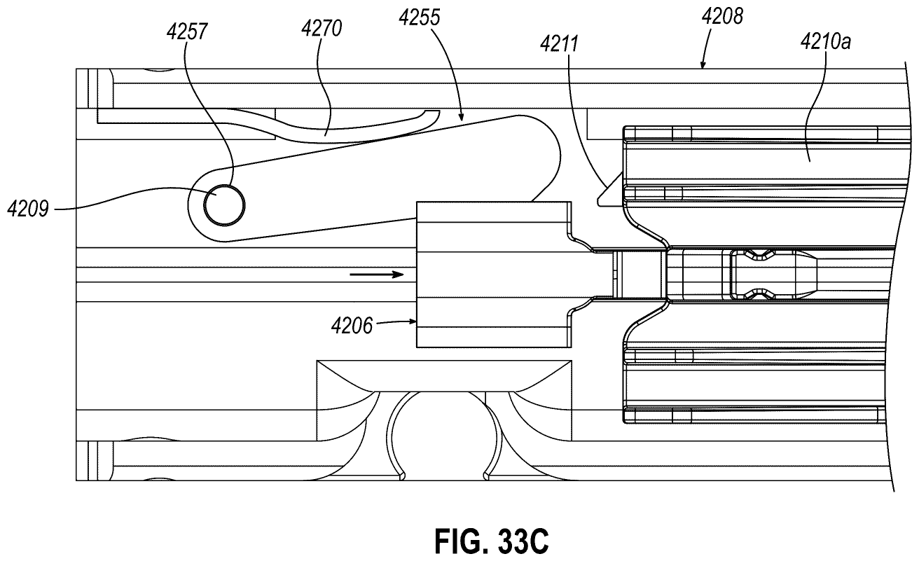

Lockout assembly 4250 includes a lockout body 4255 (also referred to herein as a “blocker”) and a lockout spring 4270. Channel 4208 includes channel post 4209 to which lockout body 4255 may be pivotally coupled. FIG. 33A shows lockout body 4255 including a post hole 4257, lockout body hook 4259 and lockout body ramp 4263. Channel post 4209 may be positioned within post hole 4257 to thereby allow lockout body 4255 to pivot. Lockout spring 4270 may be positioned between channel 4208 and lockout body 4255 to thereby apply a biasing force on lockout body 4255 to thus bias lockout body 4255 into a locked configuration as shown in FIG. 33A. Lockout body hook 4259 is positioned to engage a proximal portion of knife 4206 to thus inhibit (i.e., block) distal advancement of knife 4206. Lockout hook 4259 may be positioned to cross a knife edge plane as defined by a knife edge of knife 4206. Lockout body ramp 4263 is positioned to slidably interface with sled protrusion 4211 of sled 4210a.

FIGS. 33A-33C show the method of using lockout assembly 4250. FIG. 33A shows lockout assembly 4250 in the locked configuration and inhibiting distal translation of knife 4206. As shown in FIG. 33B, while sled 4210a is being positioned, optional sled protrusion 4211 slides against lockout body ramp 4263 to thereby transition lockout assembly 4250 into the unlocked configuration. Once in the unlocked configuration, lockout body hook 4259 no longer inhibits distal translation of knife 4206. Further, lockout spring 4270 is in a compressed configuration. FIG. 33C shows knife 4206 engaged with sled 4210a and translated beyond lockout body hook 4259. Reversing the knife 4206 from the position of FIG. 33C to the position shown in FIG. 33A may be possible by knife 4206 contacting lockout body ramp 4263 to thus rotate lockout body 4255 away such that lockout body 4255 does not inhibit travel of knife 4206 in a proximal direction. Once knife 4206 is again in the position shown in FIG. 33A, lockout assembly 4250 may again inhibit distal translation of knife 4206 until a new unspent staple cartridge having a proximally positioned sled 4210a is properly positioned (i.e., fully seated) in end effector 4200, as shown in FIG. 33B.

### E. Fifth Illustrative Firing Lockout Assembly

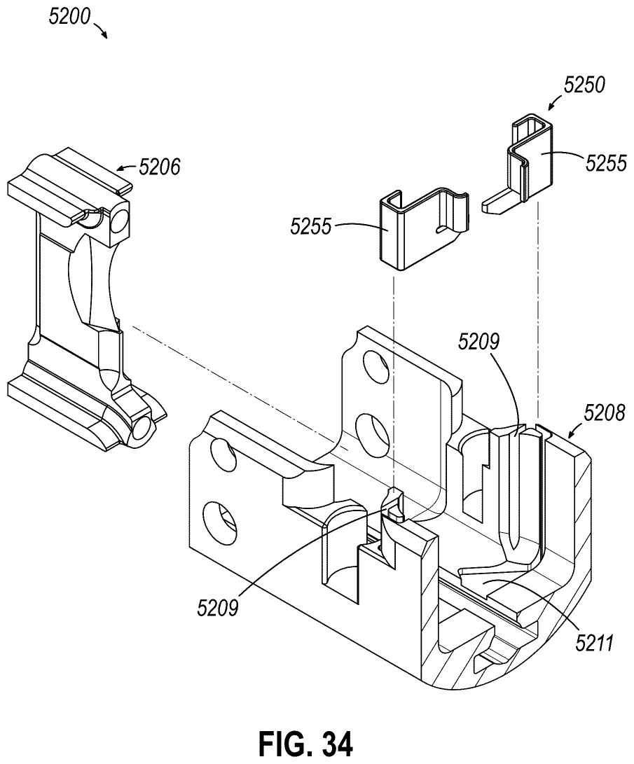

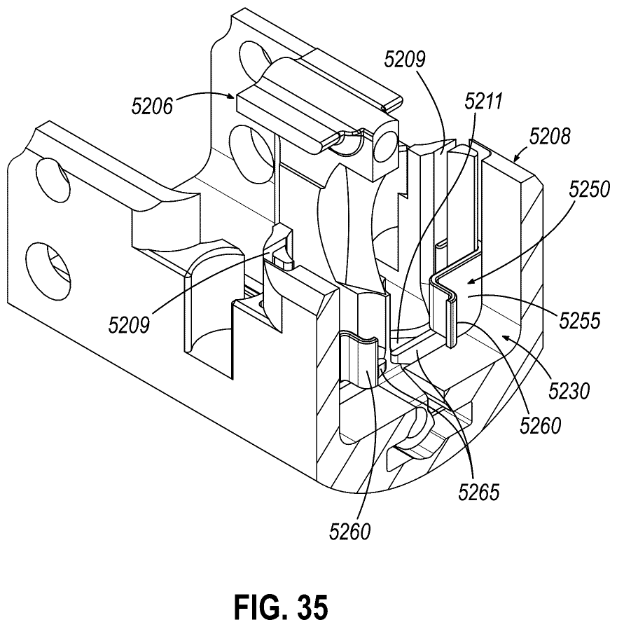

As shown in FIGS. 34-35, end effector 5200 may be substantially similar to end effectors 200, 1200, 2200, 3200, 4200 described above, except as otherwise shown and described below. End effector 5200 includes, among other features, a knife 5206 and a lockout assembly 5250. End effector 5200 further includes a cartridge jaw that is similar to cartridge jaw 202 and defines an elongate channel 5208 configured to receive a staple cartridge. As described below, lockout assembly 5250 is configured to inhibit firing of end effector 5200 in each of a spent-cartridge condition and an absent-cartridge condition, and permit firing of end effector 5200 only when an unspent staple cartridge is properly positioned (i.e., fully seated) in end effector 5200.

Lockout assembly 5250 includes two resilient lockout bodies 5255 (also referred to herein as “blockers”) positioned on opposing lateral sides of channel 5208 and having configurations that are mirrored about a vertical plane extending along a longitudinal axis of the cartridge jaw. Each lockout body 5255 includes a hooked laterally outer end that is anchored within a respective vertical slot 5209 formed in a corresponding sidewall of the cartridge jaw, and a laterally inner end having a bent sled-engagement finger 5260 that extends distally and a linear knife-engagement finger 5265 that extends laterally inward beneath sled-engagement finger 5260. As detailed below, the laterally inner end of each lockout body 5255 may resiliently deflect (i.e., flex) relative to its laterally outer end between a neutral locked position and a deflected unlocked position to thereby inhibit (i.e., block) or permit, respectively, distal translation of knife 5206 through end effector 5200. Lockout bodies 5255 are structurally independent from one another but are configured to operate in tandem, such that lockout bodies 5255 transition simultaneously between their neutral locked position and deflected unlocked position.

Each sled-engagement finger 5260 may be radiused as shown to thereby enable an underside and/or a proximal end of sled 5210a to slidably contact sled-engagement finger 5260 when an unspent staple cartridge is seated in the cartridge jaw. A lower end portion of each lockout body 5255, including knife-engagement finger 5265, is positioned within a respective opening exemplified as a recess 5211 formed in a floor of the cartridge jaw. Each recess 5211 is bounded by a proximal end shoulder wall and a distal end shoulder wall that limit an angular range of deflection of the laterally inner end of the respective lockout body 5255 relative to its laterally outer end. In particular, each knife-engagement finger 5265 is configured to abut the respective distal end shoulder wall when the lockout body 5255 is in the neutral locked position, and is configured to abut the respective proximal end shoulder wall when the lockout body 5255 is in the deflected unlocked position.

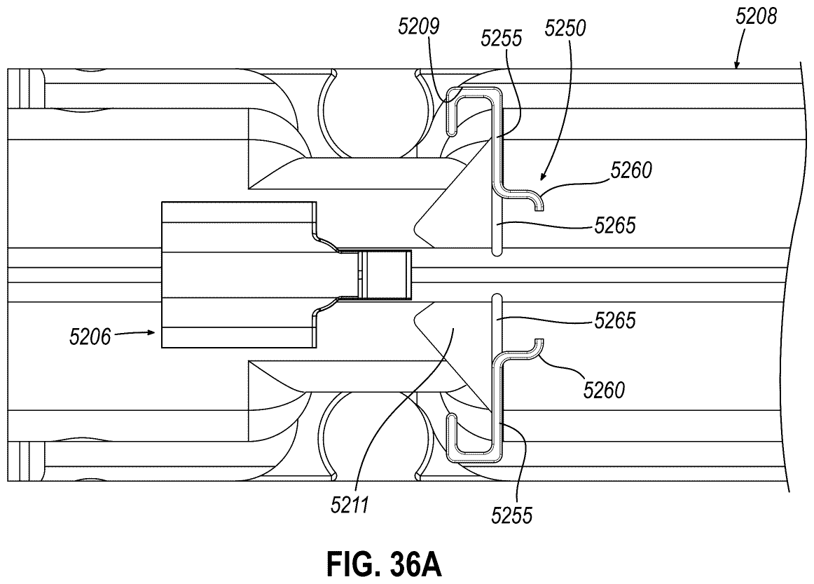

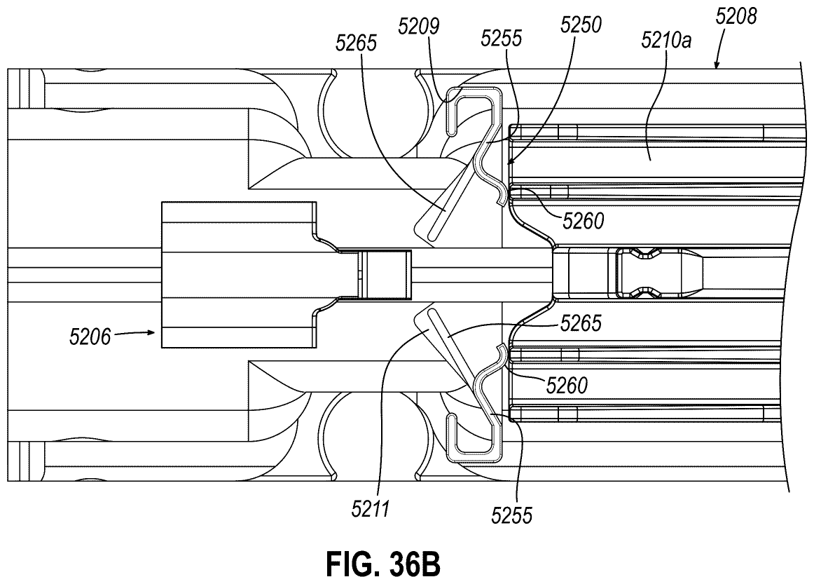

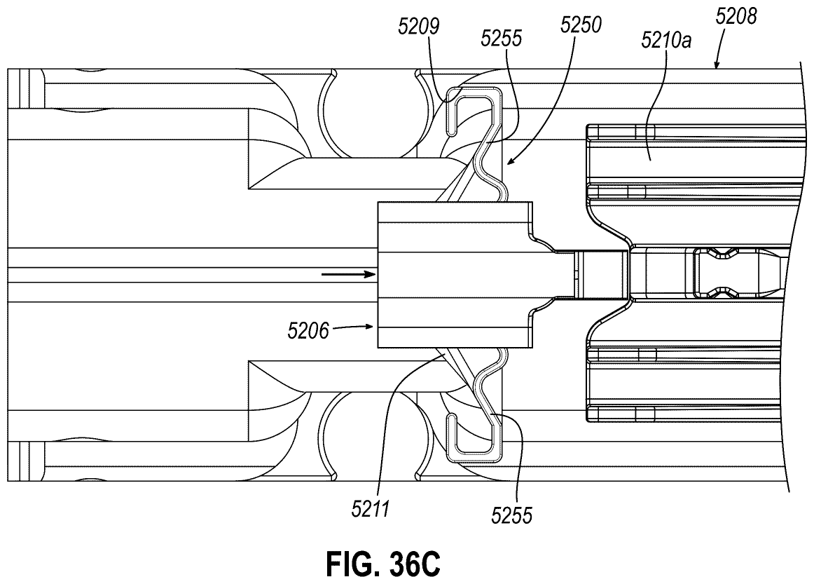

FIGS. 36A-36C show an illustrative operation of lockout assembly 5250. FIG. 36A shows knife 5206 in a proximal home position and the cartridge jaw absent of an unspent staple cartridge such that each lockout body 5255 is in its neutral locked position. In this position, knife-engagement fingers 5265 extend transversely across channel 5208 such that their terminal ends confront one another to thereby inhibit (i.e., block) distal advancement of knife 5206 through end effector 5200.

As shown in FIG. 36B, when an unspent staple cartridge is seated in the cartridge jaw of end effector 5200, a proximal end of the proximally positioned sled 5210a directly contacts and exerts a proximally-directed fore on sled-engagement fingers 5260 of lockout bodies 5255. This engagement forces the laterally inner end of each lockout body 5255, including sled-engagement finger 5260 and knife-engagement finger 5265, to angularly deflect proximally relative to its respective laterally outer end, which remains fixed in the corresponding cartidge jaw wall channel 5209, thus providing lockout bodies 5255 in the deflected unlocked positions. Knife-engagement fingers 5265 now confront the proximal shoulder walls of recesses 5211 such that their laterally inner terminal ends no longer block distal advancement of knife 5206.

As shown in FIG. 36C, with both lockout bodies 5255 in the deflected unlocked positions, knife 5206 is permitted to advance distally to engage and actuate sled 5210a through a distal firing stroke. When knife 5206 passes fully distally beyond lockout bodies 5255, lockout bodies 5255 resiliently return to their neutral locked positions. After completion of a distal firing stroke, knife 5206 may be retracted proximally so that its proximal end engages the bent knife-engagement fingers 5260 to again urge lockout bodies 5255 into deflected unlocked positions, thus permitting knife 5206 to return to its proximal home position of FIG. 36A. Once knife 5206 is again in the position shown in FIG. 36A, lockout bodies 5255 resume their neutral locked states to again inhibit distal translation of knife 5206 until a new, unspent staple cartridge having a proximally positioned sled 5210a is properly positioned (i.e., fully seated) in end effector 5200, as shown in FIG. 36B.

As described above, each lockout body 1255, 2255, 3255, 4255, 5255 may also be referred to as a “blocker.” Likewise, it will be appreciated that the term “blocker” may be substituted for the term “lockout” in referring to other components of lockout assemblies 1250, 2250 3250, 4250, 5250 as well.

### III. Examples of Combinations

The following examples relate to various non-exhaustive ways in which the teachings herein may be combined or applied. It should be understood that the following examples are not intended to restrict the coverage of any claims that may be presented at any time in this application or in subsequent filings of this application. No disclaimer is intended. The following examples are being provided for nothing more than merely illustrative purposes. It is contemplated that the various teachings herein may be arranged and applied in numerous other ways. It is also contemplated that some variations may omit certain features referred to in the below examples. Therefore, none of the aspects or features referred to below should be deemed critical unless otherwise explicitly indicated as such at a later date by the inventors or by a successor in interest to the inventors. If any claims are presented in this application or in subsequent filings related to this application that include additional features beyond those referred to below, those additional features shall not be presumed to have been added for any reason relating to patentability.

### EXAMPLE 1

An apparatus (1000) comprising: (a) a shaft (600A); and (b) an end effector (1200, 2200) operatively coupled with the shaft, wherein the end effector is configured to receive a staple cartridge (210) having a plurality of staples and a sled (1210a, 2210a) actuatable to deploy the staples into tissue clamped by the end effector, wherein the end effector includes: (i) a knife (1206, 2206) configured to cut the tissue, wherein the knife is advanceable distally through the staple cartridge to actuate the sled so that the end effector staples and cuts the tissue, (ii) an anvil jaw (204, 1204) configured to deform the staples deployed from the staple cartridge, (iii) a cartridge jaw (1208, 2208) configured to cooperate with the anvil jaw to clamp the tissue, and (iv) a blocker (1255, 2255) coupled to the cartridge jaw, wherein the blocker is vertically translatable relative to the cartridge jaw and the knife between a raised position and a lowered position, wherein in the lowered position a lower end of the blocker is positioned lower than a lower terminal end of the knife, wherein the blocker in the raised position is configured to directly contact and thereby inhibit distal advancement of the knife through the end effector, and the blocker in the lowered position is configured to permit distal advancement of the knife through the end effector, wherein the blocker is configured to assume the raised position in the absence of an unspent staple cartridge seated in the cartridge jaw and the lowered position in the presence of an unspent staple cartridge seated in the cartridge jaw.

### EXAMPLE 2

The apparatus of Example 1, wherein the blocker (1255, 2255) is configured to translate from the raised position to the lowered position in response to an unspent staple cartridge (210) being seated in the cartridge jaw.

### EXAMPLE 3

The apparatus of any of the preceding Examples, wherein the blocker (1255, 2255) includes an upwardly facing surface (1262, 2263) configured to directly contact an underside of the sled of the unspent staple cartridge when the unspent staple cartridge is seated in the cartridge jaw.

### EXAMPLE 4

The apparatus of any of the preceding Examples, wherein the blocker (1255, 2255) is biased toward the raised position such that the blocker is configured to automatically transition from the lowered position to the raised position after the knife advances distally beyond the blocker.

### EXAMPLE 5

The apparatus of Example 4, wherein the end effector further includes a blocker spring (1270, 2270) configured to resiliently bias the blocker toward the raised position.

### EXAMPLE 6

The apparatus of Example 5, wherein the blocker spring (1270, 2270) abuts a downwardly facing surface of the blocker.

### EXAMPLE 7

The apparatus of any of the preceding Examples, wherein the blocker (1255, 2255) includes a base (1280, 2280) and a pair of arms (1260, 2260) extending upwardly from the base.

### EXAMPLE 8

The apparatus of Example 7, wherein when the blocker (1255, 2255) is in the raised position the base is configured to abut the knife and thereby inhibit distal advancement of the knife, wherein when the blocker is in the lowered position the knife is configured to advance distally between the arms and over the base.

### EXAMPLE 9

The apparatus of any of the preceding Examples, wherein a lower portion of the cartridge jaw (1208, 2208) includes an opening (1209, 2209), wherein the blocker (1255, 2255) is configured to translate vertically within the opening.

### EXAMPLE 10

The apparatus of any of the preceding Examples, wherein the opening (1209, 2209) comprises a through-opening that extends through a floor of the cartridge jaw.

### EXAMPLE 11

An apparatus (1000) comprising: (a) a shaft (600A) defining a longitudinal axis; and (b) an end effector (3200) operatively coupled with the shaft, wherein the end effector is configured to receive a staple cartridge (210) having a plurality of staples and a sled (3210a) actuatable to deploy the staples into tissue clamped by the end effector, wherein the end effector includes: (i) a knife (3206) configured to cut the tissue, wherein the knife is advanceable distally through the staple cartridge to actuate the sled so that the end effector staples and cuts the tissue, (ii) an anvil jaw (204) configured to deform the staples deployed from the staple cartridge, (iii) a cartridge jaw (3208) configured to cooperate with the anvil jaw to clamp the tissue, and (iv) a blocker (3255) pivotably coupled to the cartridge jaw about a lateral pivot axis that extends transversely to the longitudinal axis, wherein the blocker is pivotable relative to the cartridge jaw and the knife between a raised position and a lowered position, wherein the blocker in the lowered position is configured to directly contact first and second lateral sides (3207) of the knife and thereby inhibit distal advancement of the knife through the end effector, and the blocker in the raised position is configured to disengage the first and second lateral sides of the knife and thereby permit distal advancement of the knife through the end effector, wherein the blocker is configured to assume the lowered position in the absence of an unspent staple cartridge seated in the cartridge jaw and the raised position in the presence of an unspent staple cartridge seated in the cartridge jaw.

### EXAMPLE 12

The apparatus of Example 11, wherein the blocker is biased toward the lowered position.

### EXAMPLE 13

The apparatus of any of Examples 11 through 12, wherein the blocker includes a pair of pivot posts (3267) that define the lateral pivot axis.

### EXAMPLE 14

The apparatus of any of Examples 11 through 13, wherein the blocker includes first and second protrusions (3265) configured to directly contact the first and second lateral sides of the knife when the blocker is in the lowered position to thereby inhibit distal advancement of the knife.

### EXAMPLE 15

The apparatus of any of Examples 11 through 14, wherein the blocker includes a pair of interconnected arms (3260), wherein the knife is configured to advance distally through a lateral gap defined between the interconnected arms when the blocker is in the raised position.

### EXAMPLE 16

An apparatus (1000) comprising: (a) a shaft (600A); and (b) an end effector (5200) operatively coupled with the shaft, wherein the end effector is configured to receive a staple cartridge (210) having a plurality of staples and a sled (5210a) actuatable to deploy the staples into tissue clamped by the end effector, wherein the end effector includes: (i) a knife (5206) configured to cut the tissue, wherein the knife is advanceable distally through the staple cartridge to actuate the sled so that the end effector staples and cuts the tissue, (ii) an anvil jaw (204) configured to deform the staples deployed from the staple cartridge, (iii) a cartridge jaw (5208) configured to cooperate with the anvil jaw to clamp the tissue, and (iv) a blocker (5255) coupled to the cartridge jaw, wherein the blocker includes a fixed laterally outer terminal end and a free laterally inner terminal end, wherein the free laterally inner terminal end is configured to resiliently deflect relative to the fixed laterally outer terminal end between a neutral position and a deflected position, wherein the blocker in the neutral position is configured to directly contact and thereby inhibit distal advancement of the knife through the end effector, and the blocker in the deflected position is configured to permit distal advancement of the knife through the end effector, wherein the blocker is configured to assume the neutral position in the absence of an unspent staple cartridge seated in the cartridge jaw and the deflected position in the presence of an unspent staple cartridge seated in the cartridge jaw.

### EXAMPLE 17

The apparatus of Example 16, wherein the blocker (5255) in the neutral position extends transversely across at least a portion of an elongate channel defined by the cartridge jaw.

### EXAMPLE 18

The apparatus of any of Examples 16 through 17, wherein the laterally inner terminal end includes a first protrusion (5260) configured to contact a sled of an unspent staple cartridge seated in the cartridge jaw, and a second protrusion (5265) configured to contact the knife.

### EXAMPLE 19

The apparatus of any of Examples 16 through 18, wherein the cartridge jaw includes a recess (5211) having end walls configured to limit an angular range of deflection of the blocker.

### EXAMPLE 20

The apparatus of any of Examples 16 through 19, wherein the blocker comprises a first blocker (5255) configured to contact a first lateral side of the knife in the neutral position, wherein the end effector further includes a second blocker (5255) laterally opposed from the first blocker and configured to contact a second lateral side of the knife in the neutral position.

The following clauses also relate to various non-exhaustive ways in which the teachings herein may be combined or applied.
1. An apparatus comprising:
   (a) a shaft; and
   (b) an end effector operatively coupled with the shaft, wherein the end effector is configured to receive a staple cartridge having a plurality of staples and a sled actuatable to deploy the staples into tissue clamped by the end effector, wherein the end effector includes:
      (i) a knife configured to cut the tissue, wherein the knife is advanceable distally through the staple cartridge to actuate the sled so that the end effector staples and cuts the tissue,
      (ii) an anvil jaw configured to deform the staples deployed from the staple cartridge,
      (iii) a cartridge jaw configured to cooperate with the anvil jaw to clamp the tissue, and
      (iv) a blocker coupled to the cartridge jaw, wherein the blocker is vertically translatable relative to the cartridge jaw and the knife between a raised position and a lowered position, wherein in the lowered position a lower end of the blocker is positioned lower than a lower terminal end of the knife,
      wherein the blocker in the raised position is configured to directly contact and thereby inhibit distal advancement of the knife through the end effector, and the blocker in the lowered position is configured to permit distal advancement of the knife through the end effector,
      wherein the blocker is configured to assume the raised position in the absence of an unspent staple cartridge seated in the cartridge jaw and the lowered position in the presence of an unspent staple cartridge seated in the cartridge jaw.
2. The apparatus of clause 1, wherein the blocker is configured to translate from the raised position to the lowered position in response to an unspent staple cartridge being seated in the cartridge jaw.
3. The apparatus of clause 1, wherein the blocker includes an upwardly facing surface configured to directly contact an underside of the sled of the unspent staple cartridge when the unspent staple cartridge is seated in the cartridge jaw.
4. The apparatus of clause 1, wherein the blocker is biased toward the raised position such that the blocker is configured to automatically transition from the lowered position to the raised position after the knife advances distally beyond the blocker.
5. The apparatus of clause 4, wherein the end effector further includes a blocker spring configured to resiliently bias the blocker toward the raised position.
6. The apparatus of clause 5, wherein the blocker spring abuts a downwardly facing surface of the blocker.
7. The apparatus of clause 1, wherein the blocker includes a base and a pair of arms extending upwardly from the base.
8. The apparatus of clause 7, wherein when the blocker is in the raised position the base is configured to abut the knife and thereby inhibit distal advancement of the knife, wherein when the blocker is in the lowered position the knife is configured to advance distally between the arms and over the base.
9. The apparatus of clause 1, wherein a lower portion of the cartridge jaw includes an opening, wherein the blocker is configured to translate vertically within the opening.
10. The apparatus of clause 1, wherein the opening comprises a through-opening that extends through a floor of the cartridge jaw.
11. An apparatus comprising:
   (a) a shaft defining a longitudinal axis; and
   (b) an end effector operatively coupled with the shaft, wherein the end effector is configured to receive a staple cartridge having a plurality of staples and a sled actuatable to deploy the staples into tissue clamped by the end effector, wherein the end effector includes:
      (i) a knife configured to cut the tissue, wherein the knife is advanceable distally through the staple cartridge to actuate the sled so that the end effector staples and cuts the tissue,
      (ii) an anvil jaw configured to deform the staples deployed from the staple cartridge,
      (iii) a cartridge jaw configured to cooperate with the anvil jaw to clamp the tissue, and
      (iv) a blocker pivotably coupled to the cartridge jaw about a lateral pivot axis that extends transversely to the longitudinal axis, wherein the blocker is pivotable relative to the cartridge jaw and the knife between a raised position and a lowered position,
      wherein the blocker in the lowered position is configured to directly contact first and second lateral sides of the knife and thereby inhibit distal advancement of the knife through the end effector, and the blocker in the raised position is configured to disengage the first and second lateral sides of the knife and thereby permit distal advancement of the knife through the end effector,
      wherein the blocker is configured to assume the lowered position in the absence of an unspent staple cartridge seated in the cartridge jaw and the raised position in the presence of an unspent staple cartridge seated in the cartridge jaw.
12. The apparatus of clause 11, wherein the blocker is biased toward the lowered position.
13. The apparatus of clause 11, wherein the blocker includes a pair of pivot posts that define the lateral pivot axis.
14. The apparatus of clause 11, wherein the blocker includes first and second protrusions configured to directly contact the first and second lateral sides of the knife when the blocker is in the lowered position to thereby inhibit distal advancement of the knife.
15. The apparatus of clause 11, wherein the blocker includes a pair of interconnected arms, wherein the knife is configured to advance distally through a lateral gap defined between the interconnected arms when the blocker is in the raised position.
16. An apparatus comprising:
   (a) a shaft; and
   (b) an end effector operatively coupled with the shaft, wherein the end effector is configured to receive a staple cartridge having a plurality of staples and a sled actuatable to deploy the staples into tissue clamped by the end effector, wherein the end effector includes:
      (i) a knife configured to cut the tissue, wherein the knife is advanceable distally through the staple cartridge to actuate the sled so that the end effector staples and cuts the tissue,
      (ii) an anvil jaw configured to deform the staples deployed from the staple cartridge,
      (iii) a cartridge jaw configured to cooperate with the anvil jaw to clamp the tissue, and
      (iv) a blocker coupled to the cartridge jaw, wherein the blocker includes a fixed laterally outer terminal end and a free laterally inner terminal end, wherein the free laterally inner terminal end is configured to resiliently deflect relative to the fixed laterally outer terminal end between a neutral position and a deflected position,
      wherein the blocker in the neutral position is configured to directly contact and thereby inhibit distal advancement of the knife through the end effector, and the blocker in the deflected position is configured to permit distal advancement of the knife through the end effector,
      wherein the blocker is configured to assume the neutral position in the absence of an unspent staple cartridge seated in the cartridge jaw and the deflected position in the presence of an unspent staple cartridge seated in the cartridge jaw.
17. The apparatus of clause 16, wherein the blocker in the neutral position extends transversely across at least a portion of an elongate channel defined by the cartridge jaw.
18. The apparatus of clause 16, wherein the laterally inner terminal end includes a first protrusion configured to contact a sled of an unspent staple cartridge seated in the cartridge jaw, and a second protrusion configured to contact the knife.
19. The apparatus of clause 16, wherein the cartridge jaw includes a recess having end walls configured to limit an angular range of deflection of the blocker.
20. The apparatus of clause 16, wherein the blocker comprises a first blocker configured to contact a first lateral side of the knife in the neutral position, wherein the end effector further includes a second blocker laterally opposed from the first blocker and configured to contact a second lateral side of the knife in the neutral position.

### IV. Miscellaneous

It should be understood that any one or more of the teachings, expressions, versions, examples, etc. described herein may be combined with any one or more of the other teachings, expressions, versions, examples, etc. that are described herein. The above-described teachings, expressions, versions, examples, etc. should therefore not be viewed in isolation relative to each other. Various suitable ways in which the teachings herein may be combined will be readily apparent to those of ordinary skill in the art in view of the teachings herein. Such modifications and variations are intended to be included within the scope of the claims.

Furthermore, any one or more of the teachings herein may be combined with any one or more of the teachings disclosed in U.S. Pat. App. No. [Atty. Ref. END9622USNP1], entitled “Surgical Stapler with Firing Lockout Feature Coupled to End Effector Knife,” filed on even date herewith; and/or U.S. Pat. App. No. [Atty. Ref. END9622USNP3], entitled “Surgical Stapler with Firing Lockout Feature Coupled to End Effector Retainer,” filed on even date herewith. The disclosure of each of the above patent references is incorporated by reference herein, in its entirety.

It should be appreciated that any patent, publication, or other disclosure material, in whole or in part, that is said to be incorporated by reference herein is incorporated herein only to the extent that the incorporated material does not conflict with existing definitions, statements, or other disclosure material set forth in this disclosure. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material.

Versions of the devices described above may have application in conventional medical treatments and procedures conducted by a medical professional, as well as application in robotic-assisted medical treatments and procedures. By way of example only, various teachings herein may be readily incorporated into a robotic surgical system such as those made available by Auris Health, Inc. of Redwood City, CA or by Intuitive Surgical, Inc., of Sunnyvale, California.

Versions of the devices described above may be designed to be disposed of after a single use, or they can be designed to be used multiple times. Versions may, in either or both cases, be reconditioned for reuse after at least one use. Reconditioning may include any combination of the steps of disassembly of the device, followed by cleaning or replacement of particular pieces, and subsequent reassembly. In particular, some versions of the device may be disassembled, and any number of the particular pieces or parts of the device may be selectively replaced or removed in any combination. Upon cleaning and/or replacement of particular parts, some versions of the device may be reassembled for subsequent use either at a reconditioning facility, or by a user immediately prior to a procedure. Those skilled in the art will appreciate that reconditioning of a device may utilize a variety of techniques for disassembly, cleaning/replacement, and reassembly. Use of such techniques, and the resulting reconditioned device, are all within the scope of the present application.

By way of example only, versions described herein may be sterilized before and/or after a procedure. In one sterilization technique, the device is placed in a closed and sealed container, such as a plastic or TYVEK bag. The container and device may then be placed in a field of radiation that can penetrate the container, such as gamma radiation, x-rays, or high-energy electrons. The radiation may kill bacteria on the device and in the container. The sterilized device may then be stored in the sterile container for later use. A device may also be sterilized using any other technique known in the art, including but not limited to beta or gamma radiation, ethylene oxide, or steam.

Having shown and described various versions of the present invention, further adaptations of the methods and systems described herein may be accomplished by appropriate modifications by one of ordinary skill in the art without departing from the scope of the present invention. Several of such potential modifications have been mentioned, and others will be apparent to those skilled in the art. For instance, the examples, versions, geometrics, materials, dimensions, ratios, steps, and the like discussed above are illustrative and are not required. Accordingly, the scope of the present invention should be considered in terms of the following claims and is understood not to be limited to the details of structure and operation shown and described in the specification and drawings.

1.-20. (canceled)

21. An apparatus comprising:
(a) a shaft; and
(b) an end effector operatively coupled with the shaft, wherein the end effector is configured to receive a staple cartridge having a plurality of staples and a sled actuatable to deploy the staples into tissue clamped by the end effector, wherein the end effector includes:
(i) a knife configured to cut the tissue, wherein the knife is advanceable distally through the staple cartridge to actuate the sled so that the end effector staples and cuts the tissue,
(ii) an anvil jaw configured to deform the staples deployed from the staple cartridge,
(iii) a cartridge jaw configured to cooperate with the anvil jaw to clamp the tissue, and
(iv) a blocker coupled to the cartridge jaw, wherein the blocker is vertically translatable relative to the cartridge jaw and the knife between a raised position and a lowered position, wherein in the lowered position a lower end of the blocker is positioned lower than a lower terminal end of the knife,
wherein the blocker in the raised position is configured to directly contact and thereby inhibit distal advancement of the knife through the end effector, and the blocker in the lowered position is configured to permit distal advancement of the knife through the end effector,
wherein the blocker is configured to assume the raised position in the absence of an unspent staple cartridge seated in the cartridge jaw and the lowered position in the presence of an unspent staple cartridge seated in the cartridge jaw.

22. The apparatus of claim 21, wherein the blocker is configured to translate from the raised position to the lowered position in response to an unspent staple cartridge being seated in the cartridge jaw.

23. The apparatus of claim 21, wherein the blocker includes an upwardly facing surface configured to directly contact an underside of the sled of the unspent staple cartridge when the unspent staple cartridge is seated in the cartridge jaw.

24. The apparatus of claim 21, wherein the blocker is biased toward the raised position such that the blocker is configured to automatically transition from the lowered position to the raised position after the knife advances distally beyond the blocker.

25. The apparatus of claim 24, wherein the end effector further includes a blocker spring configured to resiliently bias the blocker toward the raised position.

26. The apparatus of claim 25, wherein the blocker spring abuts a downwardly facing surface of the blocker.

27. The apparatus of claim 21, wherein the blocker includes a base and a pair of arms extending upwardly from the base.

28. The apparatus of claim 27, wherein when the blocker is in the raised position the base is configured to abut the knife and thereby inhibit distal advancement of the knife, wherein when the blocker is in the lowered position the knife is configured to advance distally between the arms and over the base.

29. The apparatus of claim 21, wherein a lower portion of the cartridge jaw includes an opening, wherein the blocker is configured to translate vertically within the opening.

30. The apparatus of claim 21, wherein the opening comprises a through-opening that extends through a floor of the cartridge jaw.

31. An apparatus comprising:
(a) a shaft defining a longitudinal axis; and
(b) an end effector operatively coupled with the shaft, wherein the end effector is configured to receive a staple cartridge having a plurality of staples and a sled actuatable to deploy the staples into tissue clamped by the end effector, wherein the end effector includes:
(i) a knife configured to cut the tissue, wherein the knife is advanceable distally through the staple cartridge to actuate the sled so that the end effector staples and cuts the tissue,
(ii) an anvil jaw configured to deform the staples deployed from the staple cartridge,
(iii) a cartridge jaw configured to cooperate with the anvil jaw to clamp the tissue, and
(iv) a blocker pivotably coupled to the cartridge jaw about a lateral pivot axis that extends transversely to the longitudinal axis, wherein the blocker is pivotable relative to the cartridge jaw and the knife between a raised position and a lowered position,
wherein the blocker in the lowered position is configured to directly contact first and second lateral sides of the knife and thereby inhibit distal advancement of the knife through the end effector, and the blocker in the raised position is configured to disengage the first and second lateral sides of the knife and thereby permit distal advancement of the knife through the end effector,
wherein the blocker is configured to assume the lowered position in the absence of an unspent staple cartridge seated in the cartridge jaw and the raised position in the presence of an unspent staple cartridge seated in the cartridge jaw.

32. The apparatus of claim 31, wherein the blocker is biased toward the lowered position.

33. The apparatus of claim 31, wherein the blocker includes a pair of pivot posts that define the lateral pivot axis.

34. The apparatus of claim 31, wherein the blocker includes first and second protrusions configured to directly contact the first and second lateral sides of the knife when the blocker is in the lowered position to thereby inhibit distal advancement of the knife.

35. The apparatus of claim 31, wherein the blocker includes a pair of interconnected arms, wherein the knife is configured to advance distally through a lateral gap defined between the interconnected arms when the blocker is in the raised position.

36. An apparatus comprising:
(a) a shaft; and
(b) an end effector operatively coupled with the shaft, wherein the end effector is configured to receive a staple cartridge having a plurality of staples and a sled actuatable to deploy the staples into tissue clamped by the end effector, wherein the end effector includes:
(i) a knife configured to cut the tissue, wherein the knife is advanceable distally through the staple cartridge to actuate the sled so that the end effector staples and cuts the tissue,
(ii) an anvil jaw configured to deform the staples deployed from the staple cartridge,
(iii) a cartridge jaw configured to cooperate with the anvil jaw to clamp the tissue, and
(iv) a blocker coupled to the cartridge jaw, wherein the blocker includes a fixed laterally outer terminal end and a free laterally inner terminal end, wherein the free laterally inner terminal end is configured to resiliently deflect relative to the fixed laterally outer terminal end between a neutral position and a deflected position,
wherein the blocker in the neutral position is configured to directly contact and thereby inhibit distal advancement of the knife through the end effector, and the blocker in the deflected position is configured to permit distal advancement of the knife through the end effector,
wherein the blocker is configured to assume the neutral position in the absence of an unspent staple cartridge seated in the cartridge jaw and the deflected position in the presence of an unspent staple cartridge seated in the cartridge jaw.

37. The apparatus of claim 36, wherein the blocker in the neutral position extends transversely across at least a portion of an elongate channel defined by the cartridge jaw.

38. The apparatus of claim 36, wherein the laterally inner terminal end includes a first protrusion configured to contact a sled of an unspent staple cartridge seated in the cartridge jaw, and a second protrusion configured to contact the knife.

39. The apparatus of claim 36, wherein the cartridge jaw includes a recess having end walls configured to limit an angular range of deflection of the blocker.

40. The apparatus of claim 36, wherein the blocker comprises a first blocker configured to contact a first lateral side of the knife in the neutral position, wherein the end effector further includes a second blocker laterally opposed from the first blocker and configured to contact a second lateral side of the knife in the neutral position.
